(19) United States
(12) Patent Application Publication
(10) Pub. No.: US 2010/0095403 A1
(43) Pub. Date: Apr. 15, 2010

(54) PearlI1-Like Pathogen Control Genes and Methods of use in Plants

(75) Inventors: Aaron Wiig, Chapel Hill, NC (US); Robert Ascenzi, Cary, NC (US); Xiang Huang, Apex, NC (US); Sumita Chaudhuri, Cary, NC (US); Markus Frank, Neustadt/Holstein (DE); Anton Callaway, Durham, NC (US)

Correspondence Address: BASF CORPORATION, CARL-BOSCH-STRASSE 38, LUDWIGSHAFEN, D67056, DE

(73) Assignee: BASF Plant Science Gmbh, Ludwigshafen (DE)

(21) Appl. No.: 12/525,847

(22) PCT Filed: Feb. 7, 2008
(86) PCT No.: PCT/EP2008/051483

§ 371 Date: Aug. 5, 2009

Related U.S. Application Data
(60) Provisional application No. 60/900,488, filed on Feb. 9, 2007.
(60) Provisional application No. 60/940,090, filed on May 25, 2007.

(51) Int. Cl. A01H 5/00; C12N 15/82; C07H 21/02

(52) U.S. Cl. 800/279; 800/301; 435/320.1; 536/23.1

(57) ABSTRACT

The invention provides pEARLI1-like polynucleotides which are capable of conferring increased nematode resistance to a plant. Also provided are inhibitory polynucleotides based on pEARLI1-like genes, which are capable of conferring resistance to necrotrophic fungi to plants. Specifically, the invention relates to transgenic plants, transgenic seeds, and expression vectors based on polynucleotides encoding pEARLI1-like genes, and methods of use thereof.

### CROSS REFERENCE TO RELATED APPLICATIONS

This application claims the priority benefit of U.S. Provisional Application Ser. No. 60/900,488 filed Feb. 9, 2007 and of U.S. Provisional Application Ser. No. 60/940,090 filed May 25, 2007.

### FIELD OF THE INVENTION

The invention relates to the control of biotrophic and necrotrophic plant pathogens. Disclosed herein are methods of producing transgenic plants with increased pathogen resistance, expression vectors comprising polynucleotides encoding for functional proteins, and transgenic plants and seeds generated thereof.

### BACKGROUND OF THE INVENTION

One of the major goals of plant biotechnology is the generation of plants with advantageous novel properties, for example, to increase agricultural productivity, to increase quality in the case of foodstuffs, or to produce specific chemicals or pharmaceuticals. The plant's natural defense mechanisms against pathogens are frequently insufficient. Fungal disease alone results in annual yield loses of many billions of US dollars.

Phytopathogenic fungal species generally live as saprophytes or as parasites. Parasitic fungi depend—at least during certain phases of their life cycle—on a supply of active substances (for example a supply of vitamins, carbohydrates and the like) that can only be provided by living plant cells. Experts classify some parasitic fungi as necrotrophic fungal parasites when the infection results in destruction of the tissue and thus in the death of the plant. In most cases these fungi are only facultative parasites, as they are equally capable of saprophytic growth on dead or dying plant material.

Biotrophic fungal parasites are characterized by a symbiotic relationship between parasite and host, at least over prolonged periods. While the fungus withdraws nutrients from the host, it does not kill it and may in fact prevent cell death. Most biotrophic fungi are obligate parasites. Hemibiotrophic fungi live temporarily as biotrophs and kill the host at a later point in time, i.e., they enter a necrotrophic phase.

Resistance to plant pathogenic fungi is a rather complicated and complex process. In many cases the resistance reactions start by recognition of the fungus by a plant Resistance (R-) gene product. The subsequent signaling cascade leads to hypersensitive reaction (HR), i.e., to expression of proteins that are toxic to the fungi, production of reactive oxygen species and programmed cell death. The elicitation of the HR is dependent on the activation of the salicylic acid (SA) signaling pathway. The SA-dependent HR is a powerful defense reaction to defend the plant against the attack of biotrophic pathogens, but it opens the door for the invasion of many necrotrophic pathogens, which depend on dead host tissue. To control the SA-dependent HR, plants developed a complex network of activators and inhibitors of the SA pathway. The most prominent negative modulators of the SA pathway are jasmonic acid (JA) and ethylene (ET). In addition, the SA pathway itself also modulates the activity of the JA/ET pathway. By inhibition of the HR and the activation of antimicrobial phytoalexins the JA/ET pathway is an important weapon against the attack of necrotrophic pathogens, which would benefit from host cell death.

This reciprocal regulation of resistance pathways is responsible for a balanced defense against all pathogens in wild type plants. By genetic modification, it is possible to push this balance towards resistance against the most problematic pathogen, whether it be necrotrophic or biotrophic. For example, the over-expression of a transcription factor, WRKY70, leads to enhanced resistance against Erysiphe chicoracearum by activating SA-induced genes. Simultaneously the over-expression of WRKY70 suppresses JA/ET induced genes in the plant, rendering the plant more susceptible to the necrotrophic pathogen Alternaria brassicicola.

A second example of the contrasting defense mechanisms against biotrophic and necrotrophic pathogens is the Mlo-gene. In barley, the Mlo locus has been described as a negative regulator of plant defense. The loss, or loss of function, of the Mlo gene causes an increased and, above all, species-unspecific resistance, for example, against a large number of mildew species. The knock-out of Mlo in barley results in a full penetration resistance to the biotrophic Blumeria graminis f. sp. hordei but simultaneously the barley plant becomes hypersusceptible to Magnaporthe grisea and Bipolaris sorokiniana. Additionally the overexpression of MLO leads to hypersusceptibility to Blumeria graminis f. sp. hordei.

Another large group of biotrophic plant pathogens of enormous agro-economical importance are nematodes. Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. Nematode infestation, however, can cause significant yield losses without any obvious above-ground disease symptoms. The primary causes of yield reduction are due to underground root damage. Roots infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years.

A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. However, these patents do not provide any specific nematode genes that are useful for conferring resistance to nematode infection.

A need continues to exist to identify safe and effective compositions and methods for controlling plant pathogens, and for the production of plants having increased resistance to plant pathogens.

### SUMMARY OF THE INVENTION

The present inventors have found that overexpressing pEARLI1-like genes in plants results in increased resistance to nematodes, and that inhibiting expression of pEARLI1-like genes in plants results in increased resistance to necrotrophic fungi.

Therefore, in the first embodiment, the invention provides a transgenic nematode-resistant plant transformed with an expression vector comprising an isolated pEARLI1-like polynucleotide capable of rendering a plant resistant to nematodes.

Another embodiment of the invention provides a transgenic seed which is true breeding for an expression vector comprising an isolated pEARLI1-like polynucleotide capable of rendering a plant resistant to nematodes.

Another embodiment of the invention relates to an expression cassette or an expression vector comprising a transcription regulatory element operably linked to an isolated pEARLI1-like polynucleotide capable of rendering a plant resistant to nematodes.

Another embodiment of the invention encompasses a method of producing a transgenic nematode-resistant plant, the method comprising the steps of transforming a plant cell with an expression vector comprising a transcription regulatory element operably linked to an isolated pEARLI1-like polynucleotide capable of rendering a plant resistant to nematodes, and regenerating a transgenic plant from the transformed cell.

In another embodiment, the invention provides a transgenic plant transformed with an expression vector comprising an isolated polynucleotide that inhibits expression of pEARLI1-like genes in the plant, and which render the transgenic plant resistant to necrotrophic fungi, as compared to wild type plants of the same variety.

Another embodiment of the invention provides a transgenic seed which is true breeding for an expression vector comprising an isolated polynucleotide that inhibits expression of pEARLI1-like genes, and which render the transgenic plant produced from the seed resistant to necrotrophic fungi, as compared to wild type plants of the same variety.

Another embodiment of the invention relates to an expression cassette or an expression vector comprising a transcription regulatory element operably linked to an isolated polynucleotide that inhibits expression of pEARLI1-like genes in the plant, and which render the transgenic plant resistant to necrotrophic fungi.

The invention further encompasses a dsRNA or antisense polynucleotide capable of inhibiting expression of a pEARLI1-like gene in a plant and thereby conferring resistance to a necrotrophic fungus to the plant.

### BRIEF DESCRIPTION OF THE DRAWINGS

FIG. 1 shows the table of SEQ ID NOs assigned to corresponding gene, protein and promoter sequences.

FIG. 2a-2d show the DNA and protein sequences for exemplary pEARLI1-like genes.

FIG. 3 shows an amino acid alignment of the pEARLI1-like genes At4g12500 (SEQ ID NO:2), At1g62510 (SEQ ID NO:18), At4g12490 (SEQ ID NO:10), At4g12520 (SEQ ID NO:12), At4g12530 (SEQ ID NO:20), At4g22460 (SEQ ID NO:14), and At5g46900 (SEQ ID NO:16) from Arabidopsis, and GM47093397 (SEQ ID NO:6), GM50292847 (SEQ ID NO:4), and GM50857725 (SEQ ID NO:8) from Glycine. The alignment is performed in Vector NTI software suite (gap opening penalty=10, gap extension penalty=0.05, gap separation penalty=8).

FIGS. 4a-4d show various 21mers possible for exemplary pEARLI1-like genes of SEQ ID NO:1, 3, 5, 7, 9, 11, 13, 15, 17, or 19 by nucleotide positions. For example, the 21mer could comprise nucleotides at position 1 to 21, nucleotides at position 2 to 22, nucleotides at positions 3 to 23, etc. These tables can also be used to calculate the 19, 20, 22, 23, or 24-mers by adding or subtracting the appropriate number of nucleotides from each 21mer.

FIG. 5 shows the global amino acid percent identity between pEARLI1-like genes: At4g12500 (SEQ ID NO:2), At1g62510 (SEQ ID NO:18), At4g12490 (SEQ ID NO:10), At4g12520 (SEQ ID NO:12), At4g12530 (SEQ ID NO:20), At4g22460 (SEQ ID NO:14), At5g46900 (SEQ ID NO:16), GM47093397 (SEQ ID NO:6), GM50292847 (SEQ ID NO:4), and GM50857725 (SEQ ID NO:8). Pairwise alignments and percent identities were calculated using Needle of EMBOSS-4.0.0 (Needleman, S. B. and Wunsch, C. D. (1970) J. Mol. Biol. 48, 443-453).

FIG. 6 shows the global nucleotide percent identity between pEARLI1-like genes (PLPCP genes): At4g12500 (SEQ ID NO:1), At1g62510 (SEQ ID NO:17), At4g12490 (SEQ ID NO:9), At4g12520 (SEQ ID NO:11), At4g12530 (SEQ ID NO:19), At4g22460 (SEQ ID NO:13), At5g46900 (SEQ ID NO:15), GM47093397 (SEQ ID NO:5), GM50292847 (SEQ ID NO:3), and GM50857725 (SEQ ID NO:7). Pairwise alignments and percent identities were calculated using Needle of EMBOSS-4.0.0 (Needleman, S. B. and Wunsch, C. D. (1970) J. Mol. Biol. 48, 443-453).

### DETAILED DESCRIPTION OF THE PREFERRED EMBODIMENTS

The present invention may be understood more readily by reference to the following detailed description of the embodiments of the invention and the examples included herein. Unless otherwise noted, the terms used herein are to be understood according to conventional usage by those of ordinary skill in the relevant art.

Throughout this application, various patent and scientific publications are referenced. The disclosures of all of these publications and those references cited within those publications in their entireties are hereby incorporated by reference into this application in order to more fully describe the state of the art to which this invention pertains. Abbreviations and nomenclature, where employed, are deemed standard in the field and commonly used in professional journals such as those cited herein. As used herein and in the appended claims, the singular form “a”, “an”, or “the” includes plural reference unless the context clearly dictates otherwise. As used herein, the word “or” means any one member of a particular list and also includes any combination of members of that list.

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

The polynucleotides described herein are designated pEARLI1-like polynucleotides or genes by virtue of their similarity to the Arabidopsis gene of the same name induced early in response to aluminum stress. While the function of this or of related genes is not known, it is believed that they are induced by a variety abiotic and biotic stresses. As used herein, the terms “pEARLI1-like gene” and “pEARLI1-like polynucleotides” refer to a polynucleotide having at least 70% sequence identity to any of the polynucleotides set forth in SEQ ID NOs:1, 3, 5, 7, 9, 11, 13, 15, 17, or 19; or a polynucleotide encoding a polypeptide having at least 70% sequence identity to any of the polypeptides set forth in SEQ ID NOs:2, 4, 6, 8, 10, 12, 14, 16, 18, or 20. Also encompassed in the definition of pEARLI1-like gene are homologs, orthologs, paralogs, and allelic variants of the polynucleotides set forth in SEQ ID NOs:1, 3, 5, 7, 9, 11, 13, 15, 17, or 19; or of the polynucleotide encoding a polypeptide having at least 70% sequence identity to any of the polypeptides set forth in SEQ ID NOs:2, 4, 6, 8, 10, 12, 14, 16, 18, or 20.

As used herein, the word “nucleic acid”, “nucleotide”, or “polynucleotide” is intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products.

As used herein, an “isolated” polynucleotide is substantially free of other cellular materials or culture medium when produced by recombinant techniques, or substantially free of chemical precursors when chemically synthesized.

The term “gene” is used broadly to refer to any segment of nucleic acid associated with a biological function. Thus, genes include introns and exons as in genomic sequence, or just the coding sequences as in cDNAs and/or the regulatory sequences required for their expression. For example, gene refers to a nucleic acid fragment that expresses mRNA or functional RNA, or encodes a specific protein, and which includes regulatory sequences.

The terms “polypeptide” and “protein” are used interchangeably herein to refer to a polymer of consecutive amino acid residues.

The term “operably linked” or “functionally linked” as used herein refers to the association of nucleic acid sequences on single nucleic acid fragment so that the function of one is affected by the other. For example, a regulatory DNA is said to be “operably linked to” a DNA that expresses an RNA or encodes a polypeptide if the two DNAs are situated such that the regulatory DNA affects the expression of the coding DNA.

The term “specific expression” as used herein refers to the expression of gene products that is limited to one or a few plant tissues (special limitation) and/or to one or a few plant developmental stages (temporal limitation). It is acknowledged that hardly a true specificity exists: promoters seem to be preferably switched on in some tissues, while in other tissues there can be no or only little activity. This phenomenon is known as leaky expression. However, with specific expression in this invention is meant preferable expression in one or a few plant tissues or specific sites in a plant.

The term “promoter” as used herein refers to a DNA sequence which, when ligated to a nucleotide sequence of interest, is capable of controlling the transcription of the nucleotide sequence of interest into mRNA. A promoter is typically, though not necessarily, located 5′ (e.g., upstream) of a nucleotide of interest (e.g., proximal to the transcriptional start site of a structural gene) whose transcription into mRNA it controls, and provides a site for specific binding by RNA polymerase and other transcription factors for initiation of transcription.

The term “transcription regulatory element” as used herein refers to a polynucleotide that is capable of regulating the transcription of an operably linked polynucleotide. It includes, but not limited to, promoters, enhancers, introns, 5′ UTRs, and 3′ UTRs.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. In the present specification, “plasmid” and “vector” can be used interchangeably as the plasmid is the most commonly used form of vector. A vector can be a binary vector or a T-DNA that comprises the left border and the right border and may include a gene of interest in between. The term “expression vector” as used herein means a vector capable of directing expression of a particular nucleotide in an appropriate host cell. The expression of the nucleotide can be over-expression or down-regulation. An expression vector comprises a regulatory nucleic acid element operably linked to a nucleic acid of interest, which is—optionally operably linked to a termination signal and/or other regulatory element.

As used herein, “RNAi” or “RNA interference” refers to the process of sequence-specific post-transcriptional gene silencing, mediated by double-stranded RNA (dsRNA). As used herein, “dsRNA” refers to RNA that is partially or completely double stranded. Double stranded RNA is also referred to as small or short interfering RNA (sRNA), short interfering nucleic acid (siNA), short interfering RNA, micro-RNA (miRNA), antisenseRNA, and the like. In the RNAi process, dsRNA comprising a first strand that is substantially identical to a portion of a target gene and a second strand that is complementary to the first strand is introduced into a host cell. After the introduction, the target gene-specific dsRNA is processed into relatively small fragments (siRNAs) and can subsequently become distributed throughout the host cell, leading to a loss-of-function mutation having a phenotype that, over the period of a generation, may come to closely resemble the phenotype arising from a complete or partial deletion of the target gene. Alternatively, the target gene-specific dsRNA is processed into relatively small fragments by a plant cell containing the RNAi processing machinery. A number of models have been proposed for the action of RNAi.

An “antisense” polynucleotide comprises a nucleotide sequence that is complementary to a “sense” nucleic acid encoding a protein, e.g., complementary to an mRNA sequence. Accordingly, an antisense nucleic acid can hydrogen bond to a sense nucleic acid. The antisense nucleic acid can be complementary to an entire target gene, or to a portion thereof. The antisense nucleic acid molecules are typically administered to a cell or generated in situ such that they hybridize with or bind to cellular mRNA and/or genomic DNA.

As used herein, taking into consideration of the substitution of uracil for thymine when comparing RNA and DNA sequences, the term “substantially identical” means that the nucleotide sequence of one strand of the dsRNA or antisense polynucleotide is at least 80-90% identical to 19 or more contiguous nucleotides of the target gene, or at least 90-95% identical to 19 or more contiguous nucleotides of the target gene, or at least 95-99% identical or absolutely identical to 19 or more contiguous nucleotides of the target gene.

As used herein, “complementary” polynucleotides refer to those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA.

Also as used herein, the term “substantially complementary” means that two nucleotides are complementary at least at 80% of their nucleotides, or at least at 95-90%, 90-95%, or at least at 96%, 97%, 98%, 99% or more or 100% identical of their nucleotides. Alternatively, “substantially complementary” means that two nucleotides can hybridize under high stringent conditions.

The term “homologs” as used herein refers to a gene related to a second gene by descent from a common ancestral DNA sequence. The term “homologs” may apply to the relationship between genes separated by the event of speciation (e.g., orthologs) or to the relationship between genes separated by the event of genetic duplication (e.g., paralogs).

As used herein, the term “orthologs” refers to genes from different species, but that have evolved from a common ancestral gene by speciation. Orthologs retain the same function in the course of evolution. Orthologs encode proteins having the same or similar functions. As used herein, the term “paralogs” refers to genes that are related by duplication within a genome. Paralogs usually have different functions or new functions, but these functions may be related.

As used herein, the term “allelic variant” refers to a polynucleotide containing polymorphisms that lead to changes in the amino acid sequences of a protein encoded by the nucleotide and that exist within a natural population (e.g., a plant species or variety). Such natural allelic variations can typically result in 1-5% variance in a polynucleotide encoding a protein, or 1-5% variance in the encoded protein. Allelic variants can be identified by sequencing the nucleic acid of interest in a number of different plants, which can be readily carried out by using, for example, hybridization probes to identify the same gene genetic locus in those plants.

As used herein, the term “hybridizes under stringent conditions” is intended to describe conditions for hybridization and washing under which nucleotide sequences at least 60% similar or identical to each other typically remain hybridized to each other. In another embodiment, the conditions are such that sequences at least about 65%, or at least about 70%, or at least about 75% or more similar or identical to each other typically remain hybridized to each other. Such stringent conditions are known to those skilled in the art and described as below. A preferred, non-limiting example of stringent conditions are hybridization in 6× sodium chloride/sodium citrate (SSC) at about 45° C., followed by one or more washes in 0.2×SSC, 0.1% SDS at 50-65° C.

As used herein, “percentage of sequence identity” or “sequence identity percentage” denotes a value determined by first noting in two optimally aligned sequences over a comparison window, either globally or locally, at each constituent position as to whether the identical nucleic acid base or amino acid residue occurs in both sequences, denoted a match, or does not, denoted a mismatch. As said alignment are constructed by optimizing the number of matching bases, while concurrently allowing both for mismatches at any position and for the introduction of arbitrarily-sized gaps, or null or empty regions where to do so increases the significance or quality of the alignment, the calculation determines the total number of positions for which the match condition exists, and then divides this number by the total number of positions in the window of comparison, and lastly multiplies the result by 100 to yield the percentage of sequence identity. “Percentage of sequence similarity” for protein sequences can be calculated using the same principle, wherein the conservative substitution is calculated as a partial rather than a complete mismatch. Thus, for example, where an identical amino acid is given a score of 1 and a non-conservative substitution is given a score of zero, a conservative substitution is given a score between zero and 1. The scoring of conservative substitutions can be obtained from amino acid matrices known in the art, for example, Blosum or PAM matrices.

Methods of alignment of sequences for comparison are well known in the art. The determination of percent identity or percent similarity (for proteins) between two sequences can be accomplished using a mathematical algorithm. Preferred, non-limiting examples of such mathematical algorithms are, the algorithm of Myers and Miller (Bioinformatics, 4(1):11-17, 1988), the Needleman-Wunsch global alignment (J. Mol. Biol., 48(3):443-53, 1970), the Smith-Waterman local alignment (J. Mol. Biol., 147:195-197, 1981), the search-for-similarity-method of Pearson and Lipman (PNAS, 85(8): 2444-2448, 1988), the algorithm of Karlin and Altschul (Altschul et al., J. Mol. Biol., 215(3):403-410, 1990; PNAS, 90:5873-5877, 1993). Computer implementations of these mathematical algorithms can be utilized for comparison of sequences to determine sequence identity or to identify homologs.

The term “conserved region” or “conserved domain” as used herein refers to a region in heterologous polynucleotide or polypeptide sequences where there is a relatively high degree of sequence identity between the distinct sequences. The “conserved region” can be identified, for example, from the multiple sequence alignment using the Clustal W algorithm.

The term “cell” or “plant cell” as used herein refers to single cell, and also includes a population of cells. The population may be a pure population comprising one cell type. Likewise, the population may comprise more than one cell type. A plant cell within the meaning of the invention may be isolated (e.g., in suspension culture) or comprised in a plant tissue, plant organ or plant at any developmental stage.

The term “tissue” with respect to a plant (or “plant tissue”) means arrangement of multiple plant cells, including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissues may be in planta, in organ culture, tissue culture, or cell culture.

The term “organ” with respect to a plant (or “plant organ”) means parts of a plant and may include, but not limited to, for example roots, fruits, shoots, stems, leaves, hypocotyls, cotyledons, anthers, sepals, petals, pollen, seeds, etc.

The term “plant” as used herein can, depending on context, be understood to refer to whole plants, plant cells, plant organs, plant seeds, and progeny of same. The word “plant” also refers to any plant, particularly, to seed plant, and may include, but not limited to, crop plants. Plant parts include, but are not limited to, stems, roots, shoots, fruits, ovules, stamens, leaves, embryos, meristematic regions, callus tissue, gametophytes, sporophytes, pollen, microspores, hypocotyls, cotyledons, anthers, sepals, petals, pollen, seeds and the like. The class of plants that can be used in the method of the invention is generally as broad as the class of higher and lower plants amenable to transformation techniques, including angiosperms (monocotyledonous and dicotyledonous plants), gymnosperms, ferns, horsetails, psilophytes, bryophytes, and multicellular algae.

The term “transgenic” as used herein is intended to refer to cells and/or plants which contain a transgene, or whose genome has been altered by the introduction of a transgene, or that have incorporated exogenous genes or polynucleotides. Transgenic cells, tissues, organs and plants may be produced by several methods including the introduction of a “transgene” comprising polynucleotide (usually DNA) into a target cell or integration of the transgene into a chromosome of a target cell by way of human intervention, such as by the methods described herein.

The term “true breeding” as used herein refers to a variety of plant for a particular trait if it is genetically homozygous for that trait to the extent that, when the true-breeding variety is self-pollinated, a significant amount of independent segregation of the trait among the progeny is not observed.

The term “control plant” or “wild type” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

The term “feeding site” as used herein refers to the feeding structure formed in plant roots after nematode infestation. The site is used as a source of nutrients for the nematodes. A feeding site comprises a syncytium for cyst nematodes and giant cells are comprised in the feeding sites of root knot nematodes.

The term “resistant to nematode infection” or “a plant having nematode resistance” as used herein refers to the ability of a plant to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytial or giant cells. The level of nematode resistance of a plant can be determined in various ways, e.g. by counting the nematodes being able to establish parasitism on that plant, or measuring development times of nematodes, proportion of male and female nematodes or the number of cysts or nematode eggs produced. A plant with increased resistance to nematode infection is a plant, which is more resistant to nematode infection in comparison to another plant having a similar or preferably a identical genotype while lacking the gene or genes conferring increased resistance to nematodes, e.g., a control or wild type plant.

The term “resistant to necrotrophic fungi” or “a plant having necrotrophic fungal resistance” as used herein refers to the ability of a plant to avoid infection by necrotrophic fungi, to kill necrotrophic fungi or to hamper, reduce or stop the development, growth or multiplication of necrotrophic fungi. This might be achieved by an active process, e.g. by producing a substance detrimental to the necrotrophic fungus, or by a passive process, like having a reduced nutritional value for the necrotrophic fungus. The level of necrotrophic fungus resistance of a plant can be determined, for example, such as those disclosed in Example 7 below. A plant with increased resistance to necrotrophic fungus infection is a plant, which is more resistant to necrotrophic fungus infection in comparison to another plant having a similar or preferably an identical genotype while lacking the polynucleotides conferring increased resistance to a necrotrophic fungus, e.g., a control or wild type plant.

In a first embodiment, the invention provides a transgenic nematode-resistant plant transformed with an expression vector comprising an isolated pEARLI1-like polynucleotide capable of rendering a plant resistant to nematodes. In accordance with this embodiment, the nematode-resistant transgenic plant of the invention may comprise any of the pEARLI1-like polynucleotides defined in SEQ ID NOs:1, 3, 5, 7, 9, 11, 13, 15, 17, or 19. Alternatively, the nematode-resistant transgenic plant of the invention may comprise any of the pEARLI1-like polynucleotides that encode any of the polypeptides defined in SEQ ID NOs:2, 4, 6, 8, 10, 12, 14, 16, 18, or 20. Further, the nematode-resistant transgenic plant of the invention may comprise any pEARLI1-like polynucleotide which is at least about 50-60%, or at least about 60-70%, or at least about 70-80%, 80-85%, 85-90%, 90-95%, or at least about 95%, 96%, 97%, 98%, 99% or more identical or similar to the polynucleotides defined in SEQ ID NOs:1, 3, 5, 7, 9, 11, 13, 15, 17, or 19. Moreover, the nematode-resistant transgenic plant of the invention may comprise any pEARLI1-like polynucleotide that encodes a polypeptide which is at least about 50-60%, or at least about 60-70%, or at least about 70-80%, 80-85%, 85-90%, 90-95%, or at least about 95%, 96%, 97%, 98%, 99% or more identical or similar to any of the polypeptides defined in SEQ ID NOs:2, 4, 6, 8, 10, 12, 14, 16, 18, or 20. In accordance with the invention, the nematode-resistant transgenic plant of the invention may comprise any pEARLI1-like polynucleotide that hybridizes under stringent conditions to any one of the pEARLI1-like polynucleotides defined in SEQ ID NOs:1, 3, 5, 7, 9, 11, 13, 15, 17, or 19. Alternatively, the nematode-resistant transgenic plant of the invention may comprise any pEARLI1-like polynucleotide that hybridizes under stringent conditions to any of the pEARLI1-like polynucleotides that encode any polypeptide defined in SEQ ID NOs:2, 4, 6, 8, 10, 12, 14, 16, 18, or 20.

In another embodiment, the invention provides a transgenic seed which is true breeding for an expression vector comprising an isolated pEARLI1-like polynucleotide capable of rendering a plant resistant to nematodes. In accordance with this embodiment, the transgenic seed of this embodiment may be true breeding for any of the pEARLI1-like polynucleotides defined in SEQ ID NOs:1, 3, 5, 7, 9, 11, 13, 15, 17, or 19. Alternatively, the transgenic seed of this embodiment may be true breeding for any of the pEARLI1-like polynucleotides that encode any of the polypeptides defined in SEQ ID NOs:2, 4, 6, 8, 10, 12, 14, 16, 18, or 20. Further, the transgenic seed of this embodiment may be true breeding for any pEARLI1-like polynucleotide which is at least about 50-60%, or at least about 60-70%, or at least about 70-80%, 80-85%, 85-90%, 90-95%, or at least about 95%, 96%, 97%, 98%, 99% or more identical or similar to the polynucleotides defined in SEQ ID NOs:1, 3, 5, 7, 9, 11, 13, 15, 17, or 19. Moreover, the transgenic seed of this embodiment may be true breeding for any pEARLI1-like polynucleotide that encodes a polypeptide which is at least about 50-60%, or at least about 60-70%, or at least about 70-80%, 80-85%, 85-90%, 90-95%, or at least about 95%, 96%, 97%, 98%, 99% or more identical or similar to any of the polypeptides defined in SEQ ID NOs:2, 4, 6, 8, 10, 12, 14, 16, 18, or 20. In accordance with the invention, the transgenic seed of this embodiment may be true breeding for any pEARLI1-like polynucleotide that hybridizes under stringent conditions to any one of the pEARLI1-like polynucleotides defined in SEQ ID NOs:1, 3, 5, 7, 9, 11, 13, 15, 17, or 19. Alternatively, the transgenic seed of this embodiment may be true breeding for any pEARLI1-like polynucleotide that hybridizes under stringent conditions to any of the pEARLI1-like polynucleotides that encode any polypeptide defined in SEQ ID NOs:2, 4, 6, 8, 10, 12, 14, 16, 18, or 20.

The transgenic plant or seed may be any plant or seed, such as, but not limited to trees, cut flowers, ornamentals, vegetables or crop plants. The plant may be from a genus selected from the group consisting of Medicago, Lycopersicon, Brassica, Cucumis, Solanum, Juglans, Gossypium, Malus, Vitis, Antirrhinum, Populus, Fragaria, Arabidopsis, Picea, Capsicum, Chenopodium, Dendranthema, Pharbitis, Pinus, Pisum, Oryza, Zea, Triticum, Triticale, Secale, Lolium, Hordeum, Glycine, Pseudotsuga, Kalanchoe, Beta, Helianthus, Nicotiana, Cucurbita, Rosa, Fragaria, Lotus, Medicago, Onobrychis, trifolium, Trigonella, Vigna, Citrus, Linum, Geranium, Manihot, Daucus, Raphanus, Sinapis, Atropa, Datura, Hyoscyamus, Nicotiana, Petunia, Digitalis, Majorana, Ciahorium, Lactuca, Bromus, Asparagus, Antirrhinum, Heterocallis, Nemesis, Pelargonium, Panieum, Pennisetum, Ranunculus, Senecio, Salpiglossis, Browaalia, Phaseolus, Avena, and Allium, or the plant may be selected from the group consisting of cereals including wheat, barley, sorghum, rye, triticale, maize, rice, sugarcane, and trees including apple, pear, quince, plum, cherry, peach, nectarine, apricot, papaya, mango, poplar, pine, sequoia, cedar, and oak. The term “plant” as used herein can be dicotyledonous crop plants, such as pea, alfalfa, soybean, carrot, celery, tomato, potato, cotton, tobacco, pepper, oilseed rape, beet, cabbage, cauliflower, broccoli, lettuce and Arabidopsis thaliana. In one embodiment the plant is a monocotyledonous plant or a dicotyledonous plant.

Preferably the transgenic plant or seed of the invention is a crop plant or a seed derived from a crop plant. Crop plants are all plants, used in agriculture. Accordingly in one embodiment the plant is a monocotyledonous plant, preferably a plant of the family Poaceae, Musaceae, Liliaceae or Bromeliaceae, preferably of the family Poaceae. Accordingly, in yet another embodiment the plant is a Poaceae plant of the genus Zea, Triticum, Oryza, Hordeum, Secale, Avena, Saccharum, Sorghum, Pennisetum, Setaria, Panicum, Eleusine, Miscanthus, Brachypodium, Festuca or Lolium. When the plant is of the genus Zea, the preferred species is Z. mays. When the plant is of the genus Triticum, the preferred species is T. aestivum, T. speltae or T. durum. When the plant is of the genus Oryza, the preferred species is O. sativa. When the plant is of the genus Hordeum, the preferred species is H. vulgare. When the plant is of the genus Secale, the preferred species S. cereale. When the plant is of the genus Avena, the preferred species is A. sativa. When the plant is of the genus Saccarum, the preferred species is S. officinarum. When the plant is of the genus Sorghum, the preferred species is S. vulgare, S. bicolor or S. sudanense. When the plant is of the genus Pennisetum, the preferred species is P. glaucum. When the plant is of the genus Setaria, the preferred species is S. italica. When the plant is of the genus Panicum, the preferred species is P. miliaceum or P. virgatum. When the plant is of the genus Eleusine, the preferred species is E. coracana. When the plant is of the genus Miscanthus, the preferred species is M. sinensis. When the plant is a plant of the genus Festuca, the preferred species is F. arundinaria, F. rubra or F. pratensis. When the plant is of the genus Lolium, the preferred species is L. perenne or L. multiflorum. Alternatively, the plant may be Triticosecale.

Alternatively, the transgenic plant or seed of the invention is a dicotyledonous plant, preferably a plant or seed of the family Fabaceae, Solanaceae, Brassicaceae, Chenopodiaceae, Asteraceae, Malvaceae, Linacea, Euphorbiaceae, Convolvulaceae Rosaceae, Cucurbitaceae, Theaceae, Rubiaceae, Sterculiaceae or Citrus. In one embodiment the plant is a plant of the family Fabaceae, Solanaceae or Brassicaceae. Accordingly, in one embodiment the plant is of the family Fabaceae, preferably of the genus Glycine, Pisum, Arachis, Cicer, Vicia, Phaseolus, Lupinus, Medicago or Lens. Preferred species of the family Fabaceae are M. truncatula, M, sativa, G. max, P. sativum, A. hypogea, C. arietinum, V. faba, P. vulgaris, Lupinus albus, Lupinus luteus, Lupinus angustifolius or Lens culinaris. More preferred are the species G. max A. hypogea and M. sativa. Most preferred is the species G. max. When the plant is of the family Solanaceae, the preferred genus is Solanum, Lycopersicon, Nicotiana or Capsicum. Preferred species of the family Solanaceae are S. tuberosum, L. esculentum, N. tabaccum or C. chinense. More preferred is S. tuberosum. Accordingly, in one embodiment the plant is of the family Brassicaceae, preferably of the genus Brassica or Raphanus. Preferred species of the family Brassicaceae are the species B. napus, B. oleracea, B. juncea or B. rapa. More preferred is the species B. napus. When the plant is of the family Chenopodiaceae, the preferred genus is Beta and the preferred species is the B. vulgaris. When the plant is of the family Asteraceae, the preferred genus is Helianthus and the preferred species is H. annuus. When the plant is of the family Malvaceae, the preferred genus is Gossypium or Abelmoschus. When the genus is Gossypium, the preferred species is G. hirsutum or G. barbadense and the most preferred species is G. hirsutum. A preferred species of the genus Abelmoschus is the species A. esculentus. When the plant is of the family Linacea, the preferred genus is Linum and the preferred species is L. usitatissimum. When the plant is of the family Euphorbiaceae, the preferred genus is Manihot, Jatropa or Rhizinus and the preferred species are M. esculenta, J. curcas or R. comunis. When the plant is of the family Convolvulaceae, the preferred genus is Ipomea and the preferred species is I. batatas. When the plant is of the family Rosaceae, the preferred genus is Rosa, Malus, Pyrus, Prunus, Rubus, Ribes, Vaccinium or Fragaria and the preferred species is the hybrid Fragaria x ananassa. When the plant is of the family Cucurbitaceae, the preferred genus is Cucumis, Citrullus or Cucurbita and the preferred species is Cucumis sativus, Citrullus lanatus or Cucurbita pepo. When the plant is of the family Theaceae, the preferred genus is Camellia and the preferred species is C. sinensis. When the plant is of the family Rubiaceae, the preferred genus is Coffea and the preferred species is C. arabica or C. canephora. When the plant is of the family Sterculiaceae, the preferred genus is Theobroma and the preferred species is T. cacao. When the plant is of the genus Citrus, the preferred species is C. sinensis, C. limon, C. reticulata, C. maxima and hybrids of Citrus species, or the like. In a preferred embodiment of the invention, the transgenic plant or seed is a soybean, a potato or a corn plant. In a most preferred embodiment, the transgenic plant or seed is a soybean.

The transgenic plant of the invention may be a hybrid or an inbred. The transgenic plant and seed of the invention may also be used for plant breeding, to prepare a crossed fertile transgenic plant. Suitable breeding methods are well known in agriculture, for example, a fertile transgenic plant comprising a particular expression vector of the invention may be crossed with a similar transgenic plant or with a second plant, e.g., one lacking the particular expression vector, to prepare the seed of a crossed fertile transgenic plant comprising the particular expression vector. The second plant may be an inbred plant. The seed is then planted to obtain a crossed fertile transgenic plant. The crossed fertile transgenic plant may have the particular expression vector inherited through a female parent or through a male parent. The crossed fertile transgenic may be a hybrid. Also included within the present invention are seeds of any of these crossed fertile transgenic plants.

Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The plant may be a monocot or a dicot. The crossed fertile transgenic plant may have the particular expression cassette inherited through a female parent or through a male parent. Also included within the scope of the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, specifically RNAi, by using a single transgene targeting multiple linked partial sequences of interest. Stacked, multiple genes under the control of individual promoters can also be over-expressed to attain a desired single or multiple phenotype. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired phenotypes. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

The invention is also embodied as an expression cassette or an expression vector comprising a transcription regulatory element operably linked to an isolated pEARLI1-like polynucleotide capable of rendering a plant resistant to nematodes. In accordance with this embodiment, the nematode resistance expression vector of the invention may comprise any of the pEARLI1-like polynucleotides defined in SEQ ID NOs:1, 3, 5, 7, 9, 11, 13, 15, 17, or 19. Alternatively, the nematode resistance expression vector of the invention may comprise any of the pEARLI1-like polynucleotides that encode any of the polypeptides defined in SEQ ID NOs:2, 4, 6, 8, 10, 12, 14, 16, 18, or 20. Further, the nematode resistance expression vector of the invention may comprise any pEARLI1-like polynucleotide which is at least about 50-60%, or at least about 60-70%, or at least about 70-80%, 80-85%, 85-90%, 90-95%, or at least about 95%, 96%, 97%, 98%, 99% or more identical or similar to the polynucleotides defined in SEQ ID NOs:1, 3, 5, 7, 9, 11, 13, 15, 17, or 19. Moreover, the nematode resistance expression vector of the invention may comprise any pEARLI1-like polynucleotide that encodes a polypeptide which is at least about 50-60%, or at least about 60-70%, or at least about 70-80%, 80-85%, 85-90%, 90-95%, or at least about 95%, 96%, 97%, 98%, 99% or more identical or similar to any of the polypeptides defined in SEQ ID NOs:2, 4, 6, 8, 10, 12, 14, 16, 18, or 20. In accordance with the invention, the nematode resistance expression vector may comprise any pEARLI1-like polynucleotide that hybridizes under stringent conditions to any one of the pEARLI1-like polynucleotides defined in SEQ ID NOs:1, 3, 5, 7, 9, 11, 13, 15, 17, or 19. Alternatively, the nematode resistance expression vector of the invention may comprise any pEARLI1-like polynucleotide that hybridizes under stringent conditions to any of the pEARLI1-like polynucleotides that encode any polypeptide defined in SEQ ID NOs:2, 4, 6, 8, 10, 12, 14, 16, 18, or 20.

The nematode resistance expression vector of the invention comprises one or more transcription regulatory elements operably linked to a pEARLI1-like polynucleotide capable of conferring nematode resistance to a plant. Any transcription regulatory element may be employed in the expression vectors of the invention. Preferably, the transcription regulatory element is a promoter capable of regulating constitutive expression of an operably linked polynucleotide. A “constitutive promoter” refers to a promoter that is able to express the open reading frame or the regulatory element that it controls in all or nearly all of the plant tissues during all or nearly all developmental stages of the plant. Constitutive promoters include, but are not limited to, the 35S CaMV promoter from plant viruses (Franck et al., Cell 21:285-294, 1980), the Nos promoter (An G. et al., The Plant Cell 3:225-233, 1990), the ubiquitin promoter (Christensen et al., Plant Mol. Biol. 12:619-632, 1992 and 18:581-8, 1991), the MAS promoter (Velten et al., EMBO J. 3:2723-30, 1984), the maize H3 histone promoter (Lepetit et al., Mol Gen. Genet 231:276-85, 1992), the ALS promoter (WO96/30530), the 19S CaMV promoter (U.S. Pat. No. 5,352,605), the super-promoter (U.S. Pat. No. 5,955,646), the figwort mosaic virus promoter (U.S. Pat. No. 6,051,753), the rice actin promoter (U.S. Pat. No. 5,641,876), and the Rubisco small subunit promoter (U.S. Pat. No. 4,962,028). Preferably, when the nematode resistance expression vector of the invention comprises a pEARLI1-like polynucleotide derived from G. max, the promoter is a constitutive promoter. More preferably, when the nematode resistance expression vector of the invention comprises a pEARLI1-like polynucleotide derived from G. max, the promoter is the ubiquitin promoter.

Alternatively, the promoter in the expression vector of the invention is a regulated promoter. A “regulated promoter” refers to a promoter that directs gene expression not constitutively, but in a temporally and/or spatially manner, and includes both tissue-specific and inducible promoters. Different promoters may direct the expression of a gene or regulatory element in different tissues or cell types, or at different stages of development, or in response to different environmental conditions.

A “tissue-specific promoter” or “tissue-preferred promoter” refers to a regulated promoter that is not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated, such as in early or late embryogenesis, during fruit ripening in developing seeds or fruit, in fully differentiated leaf, or at the onset of sequence. Suitable promoters include the napin-gene promoter from rapeseed (U.S. Pat. No. 5,608,152), the USP-promoter from Vicia faba (Baeumlein et al., Mol Gen Genet. 225(3):459-67, 1991), the oleosin-promoter from Arabidopsis (WO 98/45461), the phaseolin-promoter from Phaseolus vulgaris (U.S. Pat. No. 5,504,200), the Bce4-promoter from Brassica (WO 91/13980) or the legumin B4 promoter (LeB4; Baeumlein et al., Plant Journal, 2(2):233-9, 1992) as well as promoters conferring seed specific expression in monocot plants like maize, barley, wheat, rye, rice, etc. Suitable promoters to note are the Ipt2 or Ipt1-gene promoter from barley (WO 95/15389 and WO 95/23230) or those described in WO 99/16890 (promoters from the barley hordein-gene, rice glutelin gene, rice oryzin gene, rice prolamin gene, wheat gliadin gene, wheat glutelin gene, maize zein gene, oat glutelin gene, Sorghum kasirin-gene and rye secalin gene). Promoters suitable for preferential expression in plant root tissues include, for example, the promoter derived from corn nicotianamine synthase gene (US 20030131377) and rice RCC3 promoter (U.S. Ser. No. 11/075,113). Suitable promoter for preferential expression in plant green tissues include the promoters from genes such as maize aldolase gene FDA (US 20040216189), aldolase and pyruvate orthophosphate dikinase (PPDK) (Taniguchi et. al., Plant Cell Physiol. 41(1):42-48, 2000).

“Inducible promoters” refer to those regulated promoters that can be turned on in one or more cell types by an external stimulus, for example, a chemical, light, hormone, stress, or a pathogen such as nematodes. Chemically inducible promoters are especially suitable if gene expression is wanted to occur in a time specific manner. Examples of such promoters are a salicylic acid inducible promoter (WO 95/19443), a tetracycline inducible promoter (Gatz et al., Plant J. 2:397-404, 1992), the light-inducible promoter from the small subunit of Ribulose-1,5-bis-phosphate carboxylase (ssRUBISCO), and an ethanol inducible promoter (WO 93/21334). Also, suitable promoters responding to biotic or abiotic stress conditions are those such as the pathogen inducible PRP1-gene promoter (Ward et al., Plant. Mol. Biol. 22:361-366, 1993), the heat inducible hsp80-promoter from tomato (U.S. Pat. No. 5,187,267), cold inducible alpha-amylase promoter from potato (WO 96/12814), the drought-inducible promoter of maize (Busk et. al., Plant J. 11:1285-1295, 1997), the cold, drought, and high salt inducible promoter from potato (Kirch, Plant Mol. Biol. 33:897-909, 1997) or the RD29A promoter from Arabidopsis (Yamaguchi-Shinozalei et. al., Mol. Gen. Genet. 236:331-340, 1993), many cold inducible promoters such as cor15a promoter from Arabidopsis (Genbank Accession No U01377), blt101 and blt4.8 from barley (Genbank Accession Nos AJ310994 and U63993), wcs120 from wheat (Genbank Accession No AF031235), mlip15 from corn (Genbank Accession No D26563), bn115 from Brassica (Genbank Accession No U01377), and the wound-inducible pinII-promoter (European Patent No. 375091).

In one embodiment, the promoter employed in the expression vector of the invention is a root-specific, feeding site specific, pathogen inducible or nematode inducible promoter. Preferably, when the when the nematode resistance expression vector of the invention comprises a pEARLI1-like polynucleotide derived from A. thaliana, the promoter is a feeding site specific promoter. More preferably, when the when the nematode resistance expression vector of the invention comprises a pEARLI1-like polynucleotide derived from A. thaliana, the promoter is a TPP promoter having the sequence set forth in SEQ ID NO:21.

Crop plants and corresponding pathogenic nematodes are listed in Index of Plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). The nematode targeted by the present invention may be any plant parasitic nematode, like nematodes of the families Longidoridae, Trichodoridae, Aphelenchoidida, Anguinidae, Belonolaimidae, Criconematidae, Heterodidae, Hoplolaimidae, Meloidogynidae, Paratylenchidae, Pratylenchidae, Tylenchulidae, Tylenchidae, or the like. Preferably, the parasitic nematodes belong to nematode families inducing giant or syncytial cells. Nematodes inducing giant or syncytial cells are found in the families Longidoridae, Trichodoridae, Heterodidae, Meloidogynidae, Pratylenchidae or Tylenchulidae. In particular in the families Heterodidae and Meloidogynidae.

Accordingly, parasitic nematodes targeted by the present invention belong to one or more genus selected from the group of Naccobus, Cactodera, Dolichodera, Globodera, Heterodera, Punctodera, Longidorus or Meloidogyne. In a preferred embodiment the parasitic nematodes belong to one or more genus selected from the group of Naccobus, Cactodera, Dolichodera, Globodera, Heterodera, Punctodera or Meloidogyne. In a more preferred embodiment the parasitic nematodes belong to one or more genus selected from the group of Globodera, Heterodera, or Meloidogyne. In an even more preferred embodiment the parasitic nematodes belong to one or both genus selected from the group of Globodera or Heterodera. In another embodiment the parasitic nematodes belong to the genus Meloidogyne.

When the parasitic nematodes are of the genus Globodera, the species are preferably from the group consisting of G. achilleae, G. artemisiae, G. hypolysi, G. mexicana, G. millefolii, G. mali, G. pallida, G. rostochiensis, G. tabacum, and G. virginiae. In another preferred embodiment the parasitic Globodera nematodes includes at least one of the species G. pallida, G. tabacum, or G. rostochiensis. When the parasitic nematodes are of the genus Heterodera, the species may be preferably from the group consisting of H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. In another preferred embodiment the parasitic Heterodera nematodes include at least one of the species H. glycines, H. avenae, H. cajani, H. gottingiana, H. trifolii, H. zeae or H. schachtii. In a more preferred embodiment the parasitic nematodes includes at least one of the species H. glycines or H. schachtii. In a most preferred embodiment the parasitic nematode is the species H. glycines.

When the parasitic nematodes are of the genus Meloidogyne, the parasitic nematode may be selected from the group consisting of M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi. In a preferred embodiment the parasitic nematodes includes at least one of the species M. javanica, M. incognita, M. hapla, M. arenaria or M. chitwoodi.

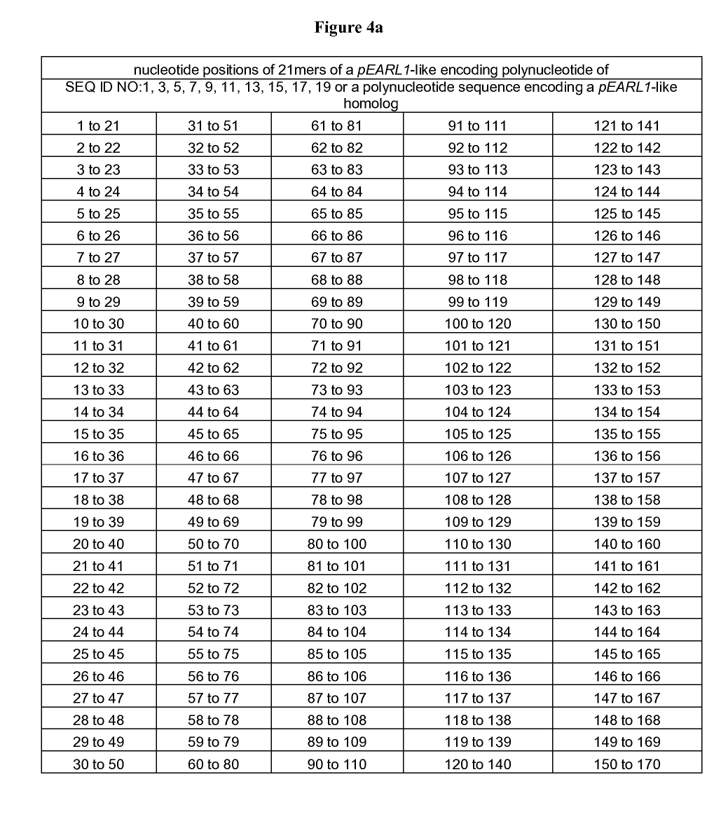

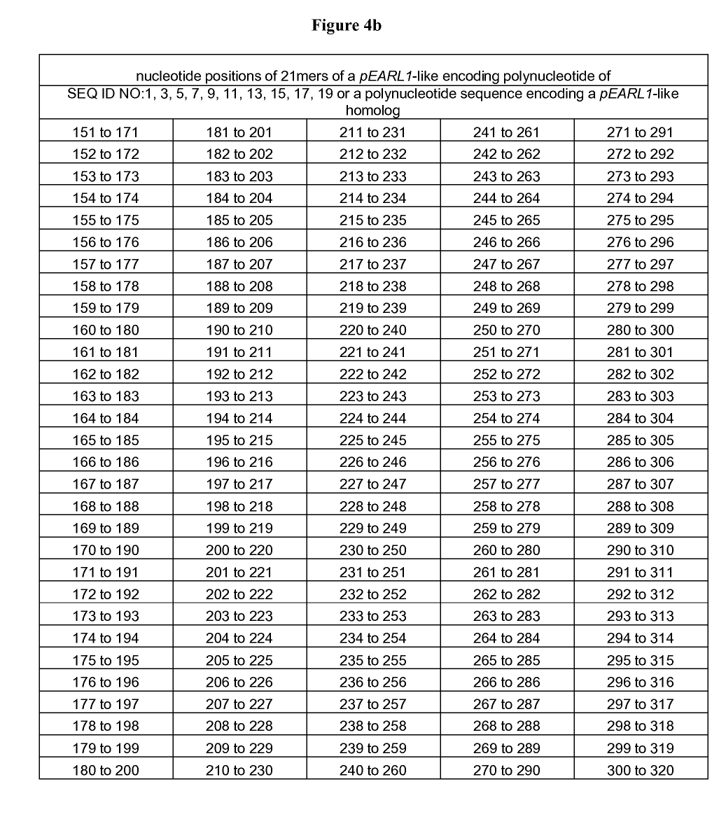

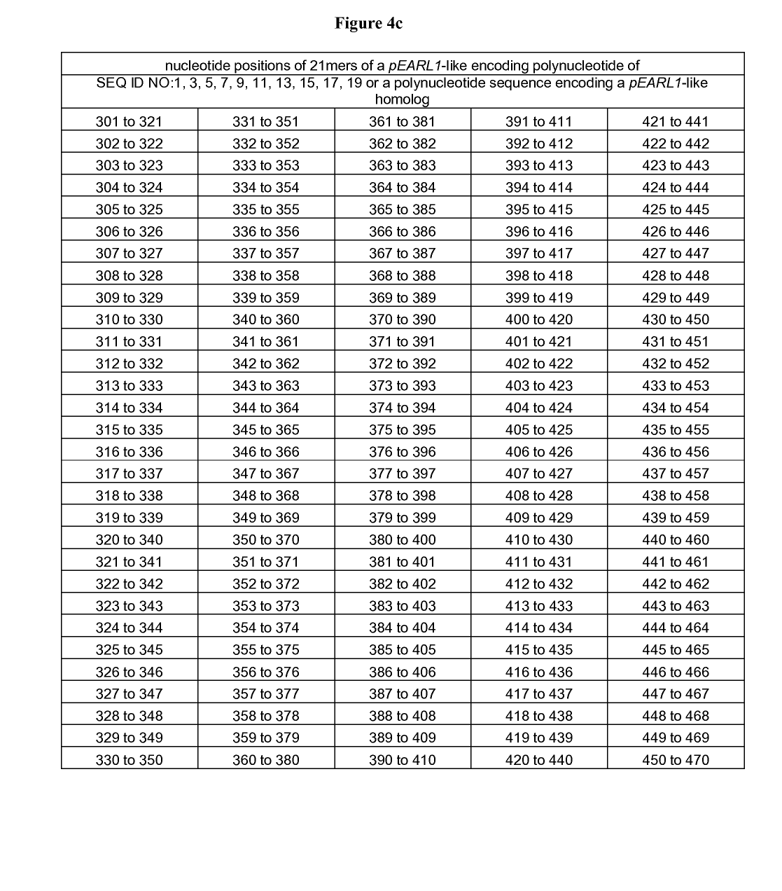

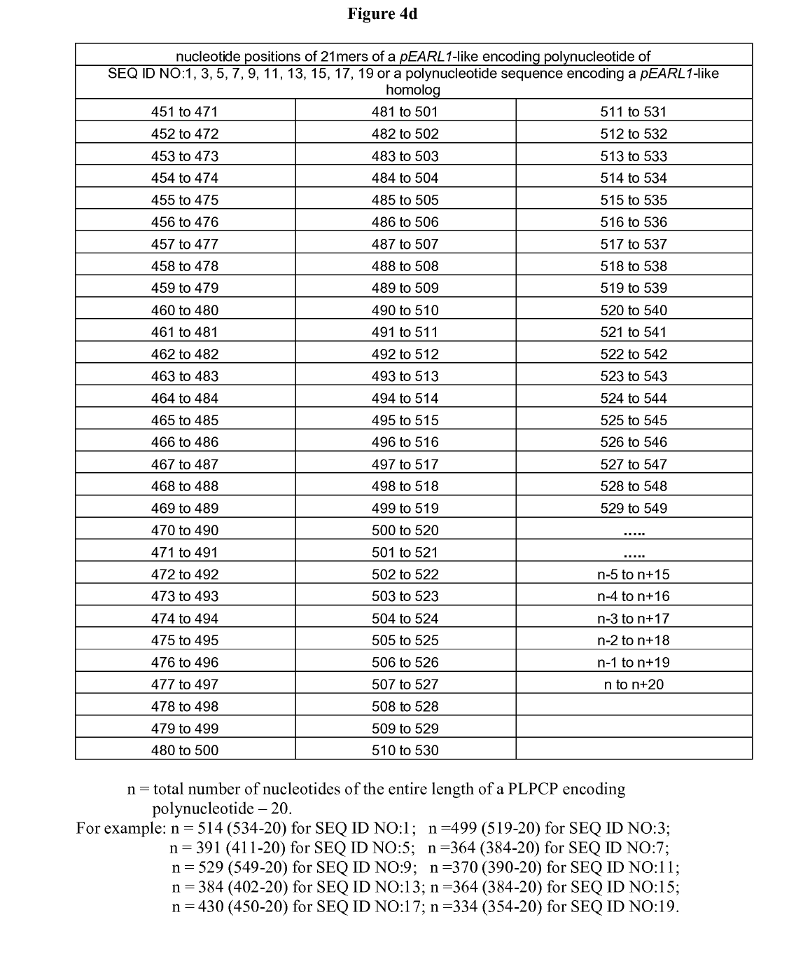

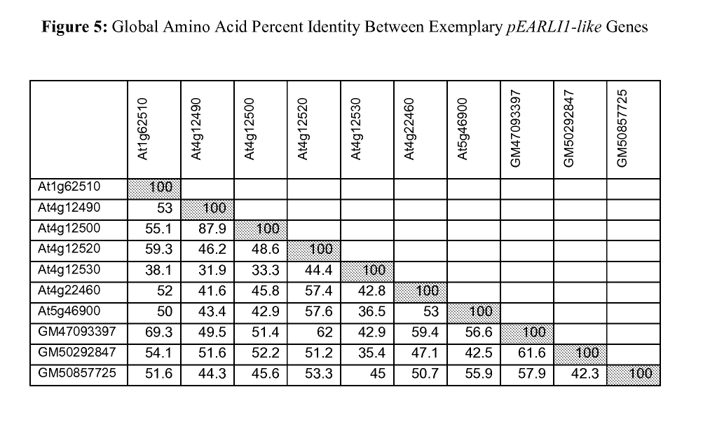

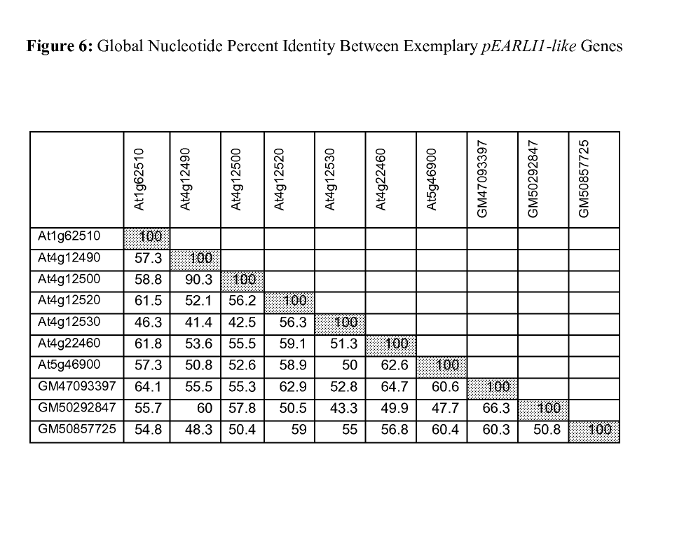

The invention is also embodied is an antifungal dsRNA or antisense polynucleotide that inhibits expression of a pEARLI1-like polynucleotide, wherein the anti-fungal comprises a first strand comprising a sequence substantially identical to a portion of a pEARLI1-like target gene. When the antifungal polynucleotide is a dsRNA, the polynucleotide further comprises a second strand that is substantially identical to the first strand. In accordance with the invention, the portion of the pEARLI1-like target gene is from 19 to 500 nucleotides of a sequence selected from the group consisting of: a) a polynucleotide sequence as defined in SEQ ID NO:1, 3, 5, 7, 9, 11, 13, 15, 17, or 19; b) a polynucleotide sequence encoding a polypeptide as defined in SEQ ID NO:2, 4, 6, 8, 10, 12, 14, 16, 18, or 20; c) a polynucleotide sequence having at least 70% sequence identity to the polynucleotide sequence defined in SEQ ID NO:1, 3, 5, 7, 9, 11, 13, 15, 17, or 19; d) a polynucleotide sequence encoding a polypeptide having at least 70% sequence identity to the polypeptide sequence defined in SEQ ID NO:2, 4, 6, 8, 10, 12, 14, 16, 18, or 20; e) a polynucleotide that hybridizes under stringent conditions to the polynucleotide defined in SEQ ID NOs:1, 3, 5, 7, 9, 11, 13, 15, 17, or 19; and f) a polynucleotide that hybridizes under stringent conditions to a polynucleotide encoding the polypeptide defined in SEQ ID NO:2, 4, 6, 8, 10, 12, 14, 16, 18, or 20. It is known that fragments of dsRNA larger than 19-24 nucleotides in length are cleaved intracellularly within eukaryotic cells to siRNAs of 19-24 nucleotides in length, and these siRNAs are the actual mediators of the RNAi phenomenon. Thus the dsRNA of the present invention may range in length from 19 nucleotides to the length of full-length target gene. Preferably, the dsRNA of the invention has a length from about 21 nucleotides to 600 nucleotides. More preferably, the dsRNA of the invention has a length from about 21 nucleotides to 500 nucleotides, or from about 21 nucleotides to 400 nucleotides. FIGS. 4a to 4d set forth exemplary 21mers derived from SEQ ID NOs:1, 3, 5, 7, 9, 11, 13, 15, 17, and 19.

The cleavage of a longer dsRNA of the invention will yield a pool of about 21mer dsRNAs (ranging from 19mers to 24mers), derived from the longer dsRNA. This pool of about 21mer dsRNAs is also encompassed within the scope of the present invention, whether generated intracellularly within the plant or nematode or synthetically using known methods of oligonucleotide synthesis.

In another embodiment, the invention provides a fungus resistance expression vector which comprises a promoter operably linked to an isolated anti-fungal dsRNA or antisense oligonucleotide that is capable of rendering a plant resistant to a fungus, preferably a necrotrophic fungus. Suitable promoters, dsRNAs, and antisense polynucleotides that may be employed in the antifungal expression vector of the invention are described above.

In yet another embodiment, the invention provides a transgenic plant comprising the fungus resistance expression vector, and seeds derived from such plants. The fungus resistant transgenic plants and seeds of this embodiment may be any of the species described above.

The transgenic fungus resistant plants of the invention may demonstrate resistance to any of the following specific fungal and oomycete pathogens. Soybeans: Phytophthora megasperma f. sp. glycinea, Phytophthora sojae, Macrophomina phaseolina, Rhizoctonia solani, Sclerotinia sclerotiorum, Fusarium oxysporum, Diaporthe phaseolorum var. sojae (Phomopsis sojae), Diaporthe phaseolorum var. caulivora, Sclerotium rolfsii, Cercospora kikuchii, Cercospora sojina, Peronospora manshurica, Colletotrichum dematium (Colletotichum truncatum), Corynespora cassuicola, Septoria glycines, Phyllosticta sojicola, Alternaria alternata, Microsphaera diffusa, Fusarium semitectum, Phialophora gregata, Glomerella glycines, Phakopsora pachyrhizi, Pythium aphanidermatum, Pythium ultimum, Pythium debaryanum, Fusarium solani f. sp. Glycines; Alfalfa: Clavibater michiganese subsp. insidiosum, Pythium ultimum, Pythium irregulare, Pythium splendens, Pythium debaryanum, Pythium aphanidermatum, Phytophthora megasperma, Peronospora trifoliorum, Phoma medicaginis var. medicaginis, Cercospora medicaginis, Pseudopeziza medicaginis, Leptotrochila medicaginis, Fusarium solani, Aphanomyces euteiches, Stemphylium herbarum, Stemphylium alfalfae; Canola: Albugo candida, Alternaria brassicae, Leptosphaeria maculans, Rhizoctonia solani, Sclerotinia sclerotiorum, Mycosphaerella brassiccola, Pythium ultimum, Peronospora parasitica, Fusarium roseum, Fusarium oxysporum, Alternaria alternate; Sunflower: Plasmophora halstedii, Sclerotinia sclerotiorum, Aster Yellows, Septoria helianthi, Phomopsis helianthi, Alternaria helianthi, Alternaria zinniae, Botrytis cinerea, Phoma macdonaldii, Macrophomina phaseolina, Erysiphe cichoracearum, Rhizopus oryzae, Rhizopus arrhizus, Rhizopus stolonifer, Puccinia helianthi, Verticillium dahliae, Erwinia carotovorum pv. carotovora, Cephalosporium acremonium, Phytophthora cryptogea, Albugo tragopogonis; Wheat: Urocystis agropyri, Alternaria alternata, Cladosporium herbarum, Fusarium graminearum, Fusarium avenaceum, Fusarium culmorum, Ustilago tritici, Ascochyta tritici, Cephalosporium gramineum, Collotetrichum graminicola, Erysiphe graminis f. sp. tritici, Puccinia graminis f. sp. tritici, Puccinia recondita f. sp. tritici, Puccinia striiformis, Pyrenophora tritici-repentis, Septoria nodorum, Septoria tritici, Septoria avenae, Pseudocercosporella herpotrichoides, Rhizoctonia solani, Rhizoctonia cerealis, Gaeumannomyces graminis var. tritici, Pythium aphanidermatum, Pythium arrhenomanes, Pythium ultimum, Bipolaris sorokiniana, Claviceps purpurea, Tilletia tritici, Tilletia laevis, Ustilago tritici, Tilletia indica, Rhizoctonia solani, Pythium arrhenomannes, Pythium gramicola, Pythium aphanidermatum; Corn: Fusarium moniliforme var. subglutinans, Fusarium moniliforme, Gibberella zeae (Fusarium graminearum), Stenocarpella maydi (Diplodia maydis), Pythium irregulare, Pythium debaryanum, Pythium graminicola, Pythium splendens, Pythium ultimum, Pythium aphanidermatum, Aspergillus flavus, Bipolaris maydis O, T (Cochliobolus heterostrophus), Helminthosporium carbonum I, II & III (Cochliobolus carbonum), Exserohilum turcicum I, II & III, Helminthosporium pedicellatum, Physoderma maydis, Phyllosticta maydis, Kabatiella maydis, Cercospora sorghi, Ustilago maydis, Puccinia sorghi, Puccinia polysora, Macrophomina phaseolina, Penicillium oxalicum, Nigrospora oryzae, Cladosporium herbarum, Curvularia lunata, Curvularia inaequalis, Curvularia pallescens, Clavibacter michiganense subsp. nebraskense, Trichoderma viride, Claviceps sorghi, Corn stunt spiroplasma, Diplodia macrospora, Sclerophthora macrospora, Peronosclerospora sorghi, Peronosclerospora philippinensis, Peronosclerospora maydis, Peronosclerospora sacchari, Sphacelotheca reiliana, Physopella zeae, Cephalosporium maydis, Cephalosporium acremonium; Sorghum: Exserohilum turcicum, Colletotrichum graminicola (Glomerella graminicola), Cercospora sorghi, Gloeocercospora sorghi, Ascochyta sorghina, Puccinia purpurea, Macrophomina phaseolina, Perconia circinata, Fusarium moniliforme, Alternaria alternata, Bipolaris sorghicola, Helminthosporium sorghicola, Curvularia lunata, Phoma insidiosa, Ramulispora sorghi, Ramulispora sorghicola, Phyllachara sacchari, Sporisorium reilianum (Sphacelotheca reiliana), Sphacelotheca cruenta, Sporisorium sorghi, Rhizoctonia solani, Acremonium strictum, Sclerophthona macrospora, Peronosclerospora sorghi, Peronosclerospora philippinensis, Sclerospora graminicola, Fusarium graminearum, Fusarium oxysporum, Pythium arrhenomanes, Pythium graminicola, etc. (U.S. Pat. No. 6,630,618)

The fungal or oomycete pathogen may be a necrotrophic or at least partially necrotrophic fungal or oomycete fungus. Preferably the fungal or oomycete pathogen is a necrotrophic fungal or oomycete pathogen. T

Another embodiment of the invention encompasses a method of producing a transgenic nematode-resistant plant, the method comprising the steps of transforming a plant cell with a nematode resistance expression vector comprising a promoter operably linked to an isolated pEARLI1-like polynucleotide capable of rendering a plant resistant to nematodes, regenerating a transgenic plant from the transformed cell, and selecting regenerated transgenic plants for increased nematode resistance.

Another embodiment of the invention encompasses a method of producing a transgenic fungus-resistant plant, the method comprising the steps of transforming a plant cell with a fungal resistance expression vector comprising a promoter operably linked to an dsRNA or antisense oligonucleotide that targets a pEARLI1-like gene, wherein the dsRNA or antisense polynucleotide is capable of rendering a plant resistant to fungus, regenerating a transgenic plant from the transformed cell, and selecting regenerated transgenic plants for increased fungal resistance.

A variety of methods for introducing polynucleotides into the genome of plants and for the regeneration of plants from plant tissues or plant cells are known in, for example, Plant Molecular Biology and Biotechnology (CRC Press, Boca Raton, Fla.), chapter 6/7, pp. 71-119 (1993); White F F (1993) Vectors for Gene Transfer in Higher Plants; Transgenic Plants, vol. 1, Engineering and Utilization, Ed.: Kung and Wu R, Academic Press, 15-38; Jenes B et al. (1993) Techniques for Gene Transfer; Transgenic Plants, vol. 1, Engineering and Utilization, Ed.: Kung and R. Wu, Academic Press, pp. 128-143; Potrykus (1991) Annu Rev Plant Physiol Plant Molec Biol 42:205-225; Halford N G, Shewry P R (2000) Br Med Bull 56(1):62-73.

Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm M E et al., Bio/Technology. 8(9):833-9, 1990; Gordon-Kamm et al. Plant Cell 2:603, 1990), electroporation, incubation of dry embryos in DNA-comprising solution, and microinjection. In the case of these direct transformation methods, the plasmids used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229. The Agrobacterium-mediated transformation is best suited to dicotyledonous plants but has also been adapted to monocotyledonous plants. The transformation of plants by Agrobacteria is described in, for example, White F F, Vectors for Gene Transfer in Higher Plants, Transgenic Plants, Vol. 1, Engineering and Utilization, edited by S. D. Kung and R. Wu, Academic Press, 1993, pp. 15-38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225.

Transformation may result in transient or stable transformation and expression. Although a nucleotide sequence of the present invention can be inserted into any plant and plant cell falling within these broad classes, it is particularly useful in crop plant cells. The nucleotides of the present invention can be directly transformed into the plastid genome. Plastid expression, in which genes are inserted by homologous recombination into the several thousand copies of the circular plastid genome present in each plant cell, takes advantage of the enormous copy number advantage over nuclear-expressed genes to permit high expression levels. In one embodiment, the nucleotides are inserted into a plastid targeting vector and transformed into the plastid genome of a desired plant host. Plants homoplasmic for plastid genomes containing the nucleotide sequences are obtained, and are preferentially capable of high expression of the nucleotides.

Plastid transformation technology is for example extensively described in U.S. Pat. Nos. 5,451,513, 5,545,817, 5,545,818, and 5,877,462 in WO 95/16783 and WO 97/32977, and in McBride et al. (1994) PNAS 91, 7301-7305, all incorporated herein by reference in their entirety. The basic technique for plastid transformation involves introducing regions of cloned plastid DNA flanking a selectable marker together with the nucleotide sequence into a suitable target tissue, e.g., using biolistic or protoplast transformation (e.g., calcium chloride or PEG mediated transformation). The 1 to 1.5 Kb flanking regions, termed targeting sequences, facilitate homologous recombination with the plastid genome and thus allow the replacement or modification of specific regions of the plastome. Initially, point mutations in the chloroplast 16S rRNA and rps12 genes conferring resistance to spectinomycin and/or streptomycin are utilized as selectable markers for transformation (Svab et al., PNAS 87, 8526-8530, 1990; Staub et al., Plant Cell 4, 39-45, 1992). The presence of cloning sites between these markers allows creation of a plastid targeting vector for introduction of foreign genes (Staub et al. EMBO J. 12, 601-606, 1993). Substantial increases in transformation frequency are obtained by replacement of the recessive rRNA or r-protein antibiotic resistance genes with a dominant selectable marker, the bacterial aadA gene encoding the spectinomycin-detoxifying enzyme aminoglycoside-3′-adenyltransferase (Svab et al., PNAS 90, 913-917, 1993). Other selectable markers useful for plastid transformation are known in the art and encompassed within the scope of the invention.

The transgenic plants of the invention may be used in a method of controlling infestation of a crop by a plant pathogen, which comprises the step of growing said crop from seeds comprising an expression vector comprising one or more transcription regulatory elements operably linked to one or more polynucleotides that encode an agent toxic to said plant pathogen, wherein the expression vector is stably integrated into the genomes of the seeds.

While the compositions and methods of this invention have been described in terms of certain embodiments, it will be apparent to those of skilled in the art that variations may be applied to the composition, methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

### Example 1

### Identification of pEARLI1-Like Genes in SCN-Infected Roots

Microarray analysis of laser excised syncytial cells of soybean roots inoculated with second-stage juveniles (J2) of H. glycines race3 led to the identification of genes expressed specifically or differentially in syncytia. Three such soybean genes (corresponding to cDNA clones GM50292847, GM47093397, and GM50857725) were down-regulated in the syncytia compared to uninfected root tissue as shown in Table 1, which summarizes the expression data as measured by cDNA microarray analysis across all three cell/tissue samples: syncytia, SCN infected non-syncytia and untreated control root tissues. Relative levels of gene expression are expressed as normalized signal intensities (±standard deviation).

TABLE 1
Expression of pEARLI1-like soybean genes

| Gene Name | Syncytia #1 (N) | Syncytia #2 (N) | Non-Syncytia | Control Roots |
|---|---|---|---|---|
| GM50292847 | ND** | 90 ± 37 | 92 ± 39 | 410 ± 278 |
| GM47093397 | 63 ± 18 | 133 ± 97 | 222 ± 58 | 2122 ± 1798 |
| GM50857725 | ND | ND | 146 ± 98 | 728 ± 443 |

**Not detectable under experimental conditions described in this study

### Example 2

### Cloning of pEARLI1-Like Gene At4g12500

The Arabidopsis pEARLI1-like gene encoded by At4g12500 was selected based on its similarity to the soybean cDNA sequences indicated in Example 1. In order to express this protein the coding sequence was PCR amplified from genomic DNA, which lacks introns, using the standard molecular biology techniques. The amplified product was ligated into a TOPO entry vector (Invitrogen, Carlsbad, Calif.).

### Example 3

### Vector Construction for Transformation and Generation of Transgenic Roots

The cloned coding region from At4g12500 pEARLI1-like generated in Example 2 was sequenced and subcloned into a plant expression vector containing a syncytia-preferred (nematode-induced) promoter using the Gateway™ system. The syncytia-preferred TPP-like promoter (SEQ ID NO: 21, U.S. Ser. No. 60/874,375) was used in the construct as shown in Table 2. The selection marker for transformation was BAR, a gene that conferred resistance to the herbicide LIBERTY (glufosinate, Bayer Crop Science, Kansas City, Mo., US).

TABLE 2
Expression vector comprising SEQ ID NO: 1

| | Composition of the over-expression vector |
|---|---|
| vector | (promoter::pEARLI1-like polynucleotide) |
| RLM565 | TPP-like promoter:: At4g12500 |

### Example 4

### Use of Soybean Plant Assay System to Detect Resistance to SCN Infection

The proprietary rooted explant assay was employed to demonstrate over-expression of pEARLI1-like genes and the resulting nematode resistance. This assay can be found in commonly owned co-pending application U.S. Ser. No. 12,001,234.

Clean soybean seeds from soybean cultivar were surface sterilized and germinated. Three days before inoculation, an overnight liquid culture of the disarmed Agrobacterium culture, for example, the disarmed A. rhizogenes strain K599 containing the binary vector RLM565 was initiated. The next day the culture was spread onto an LB agar plate containing kanamycin as a selection agent. The plates were incubated at 28° C. for two days. One plate was prepared for every 50 explants to be inoculated. Cotyledons containing the proximal end from its connection with the seedlings were used as the explant for transformation. After removing the cotyledons the surface was scraped with a scalpel around the cut site. The cut and scraped cotyledon was the target for Agrobacterium inoculation. The prepared explants were dipped onto the disarmed thick A. rhizogenes colonies prepared above so that the colonies were visible on the cut and scraped surface. The explants were then placed onto 1% agar in Petri dishes for co-cultivation under light for 6-8 days.

After the transformation and co-cultivation soybean explants were transferred to rooting induction medium with a selection agent, for example S-B5-708 for the mutated acetohydroxy acid synthase (AHAS) gene (Sathasivan et al., Plant Phys. 97:1044-50, 1991). Cultures were maintained in the same condition as in the co-cultivation step. The S-B5-708 medium comprises: 0.5× B5 salts, 3 mM MES, 2% sucrose, 1× B5 vitamins, 400 μg/ml Timentin, 0.8% Noble agar, and 1 μM Imazapyr (selection agent for AHAS gene) (BASF Corporation, Florham Park, N.J.) at pH 5.8.

Two to three weeks after the selection and root induction, transformed roots were formed on the cut ends of the explants. Explants were transferred to the same selection medium (S-B5-708 medium) for further selection. Transgenic roots proliferated well within one week in the medium and were ready to be subcultured. Strong and white soybean roots were excised from the rooted explants and cultured in root growth medium supplemented with 200 mg/l Timentin (S-MS-606 medium) in six-well plates. Cultures were maintained at room temperature under the dark condition. The S-MS-606 medium comprises: 0.2×MS salts and B5 vitamins, 2% sucrose, and 200 mg/l Timentin at pH 5.8.

One to five days after subculturing, the roots were inoculated with surface sterilized nematode juveniles in multi-well plates for either gene of interest or promoter construct assay. Soybean cultivar Williams 82 control vector and Jack control vector roots were used as susceptible and resistant controls, respectively. Transformed root cultures of each line were inoculated with surface-decontaminated race 3 of soybean cyst nematode (SCN) second stage juveniles (J2) at the level of 500 J2/well. The plates were sealed and maintained at 25° C. in darkness. Several independent root lines were generated from each binary vector transformation and the lines were used for bioassay.

Four weeks after nematode inoculation, the cyst number in each well was counted. For each transformed line, the average number of cysts per line, the female index and the standard error values were determined across several replicated wells (Female index=average number of SCN cysts developing on the transgenic roots expressed as percentage of the average number of cysts developing on the W82 wild type susceptible control roots). The results show that the majority of RLM565 transformed roots had reduced cyst counts over multiple transgenic lines and a general trend of reduced cyst count in the majority of transgenic lines assayed, relative to the susceptible control roots.

### Example 5

### Identifying Additional pEARLI1-Like Genes and Cloning into Binary Expression Vectors

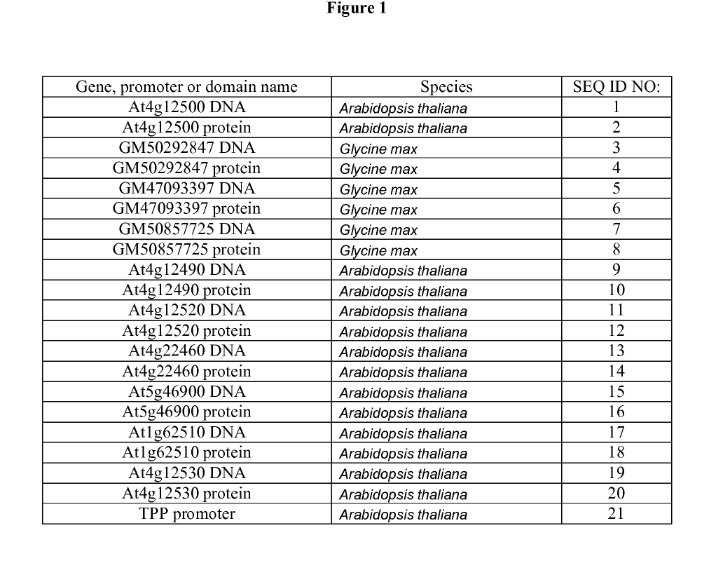

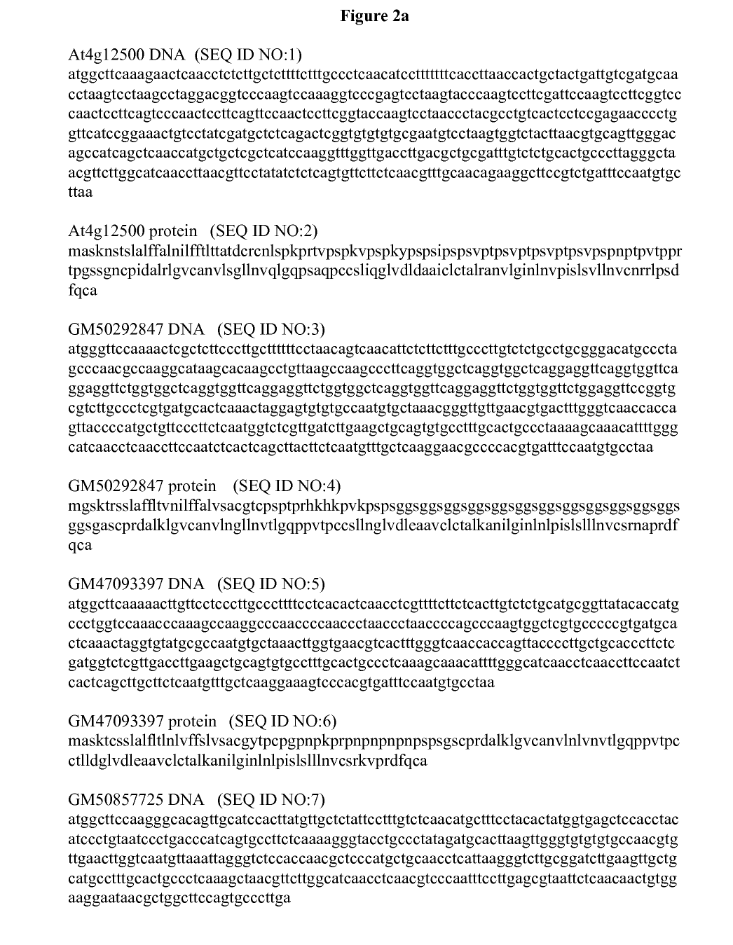

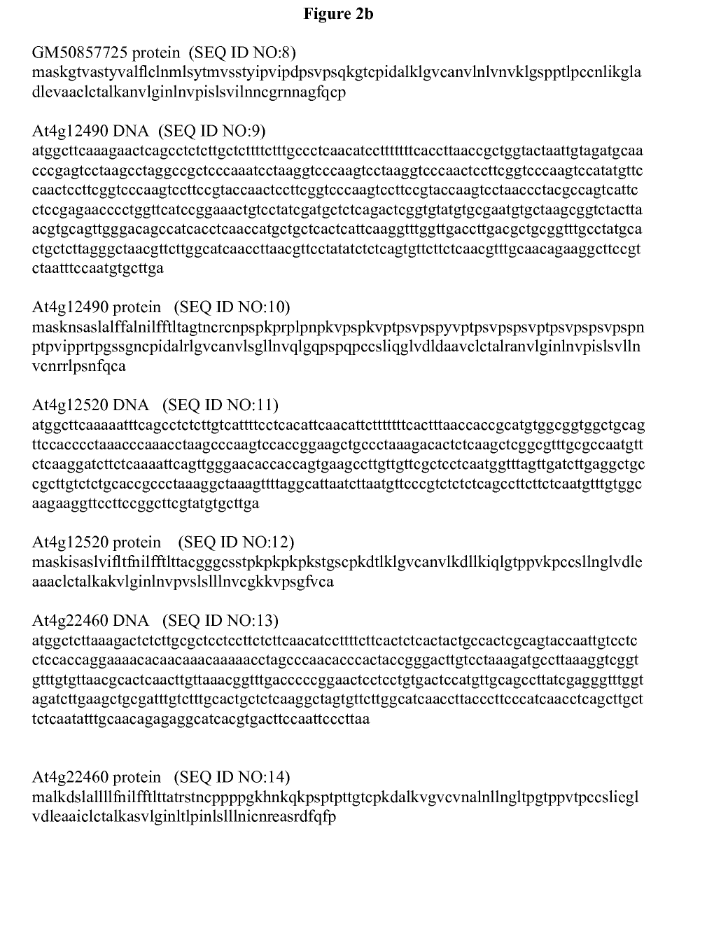

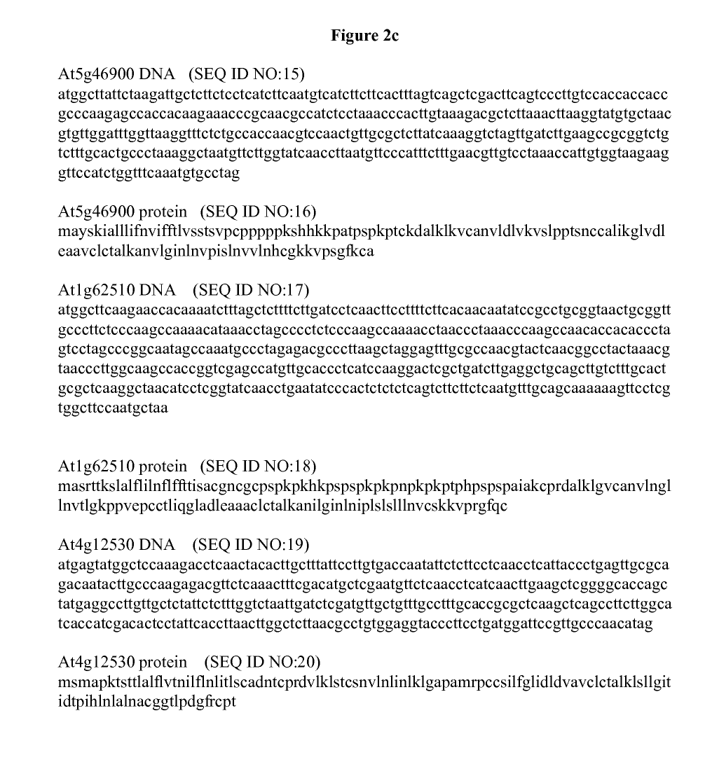

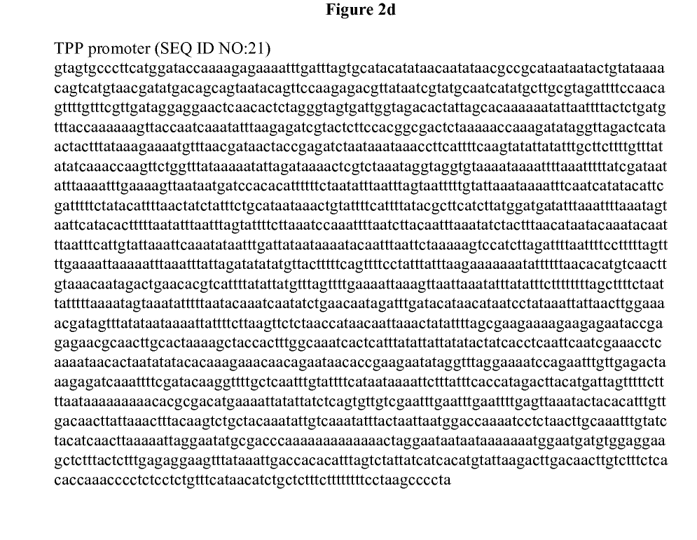

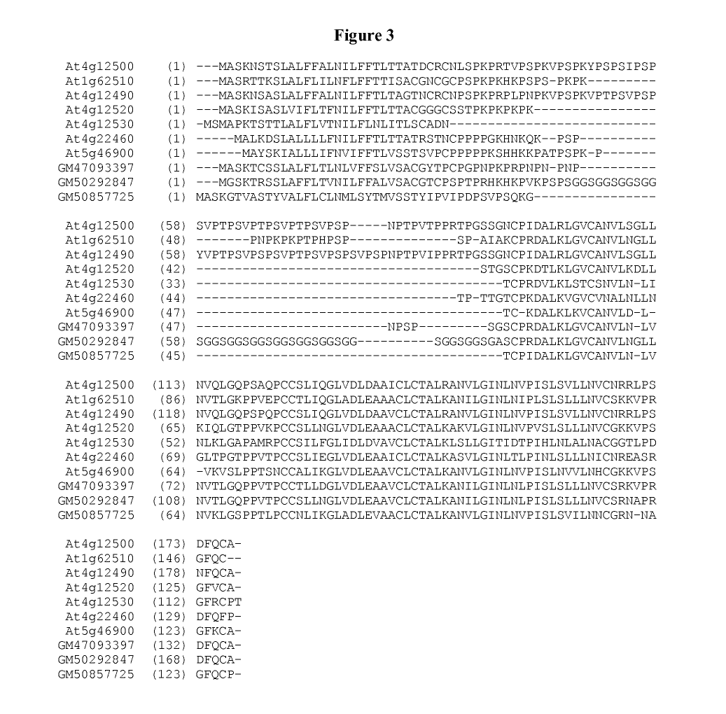

Additional pEARLI1-like genes were identified based on nucleotide and protein sequence similarity to At4g12500 (SEQ ID NO: 1 and SEQ ID NO: 2, respectively) by performing BLAST searches against proprietary cDNA databases and public databases, such as Genbank, TIGR and TAIR. Some of the pEARLI1-like genes thus identified are listed in FIG. 1.

The full length coding region of soybean GM50292847 (SEQ ID NO: 3) was PCR amplified from the proprietary cDNA clone 50292847. The PCR product was cloned into an intermediate TOPO-TA vector (Invitrogen, Carlsbad, Calif.), sequenced and then subcloned into a plant binary expression vector using standard restriction digest and ligation reactions. The resulting vector, hereafter referred to as RBM020 or pBM020, contained the GM50292847 coding sequence expressed under the control of the ubiquitin promoter from parsley (WO 03/102198). The selection marker for transformation was the mutated form of the AHAS selection gene (also referred to as AHAS2) from Arabidopsis thaliana (Sathasivan et al., Plant Phys. 97:1044-50, 1991), conferring resistance to the herbicide ARSENAL (imazapyr, BASF Corporation, Mount Olive, N.J.). Expression of the AHAS2 selection marker was also controlled by the parsley ubiquitin promoter.

Full length coding regions from Arabidopsis At4g22460 (SEQ ID NO: 13), At1g62510 (SEQ ID NO:17), At4g12530 (SEQ ID NO:19), At4g12490 (SEQ ID NO: 9), At4g12520 (SEQ ID NO: 11) and At5g46900 (SEQ ID NO:15), all of which lack introns, were PCR amplified from Arabidopsis thaliana, ecotype Col-0 genomic DNA. The amplified regions were cloned into plant binary expression vectors under the control of the TPP-like promoter (SEQ ID NO:21) with the AHAS2 selection marker described above. The vector names corresponding to specific pEARLI1-like expression constructs are shown in Table 3 below.

TABLE 3
Soybean and Arabidopsis pEARLI1-like expression vectors

| | Composition of the expression vector | SEQ ID NO for coding |
|---|---|---|
| vector | (promoter::PLPCP) | sequence |
| RBM020 | Ubiquitin promoter::GM50292847 | SEQ ID NO: 3 |
| RCB873 | TPP-like promoter::At4g22460 | SEQ ID NO: 13 |
| RCB868 | TPP-like promoter::At1g62510 | SEQ ID NO: 17 |
| RCB872 | TPP-like promoter::At4g12530 | SEQ ID NO: 19 |
| RCB869 | TPP-like promoter::At4g12490 | SEQ ID NO: 9 |
| RCB871 | TPP-like promoter::At4g12520 | SEQ ID NO: 11 |
| RCB874 | TPP-like promoter::At5g46900 | SEQ ID NO: 15 |

### Example 6

### Nematode Assay for RBM020, RCB873 and RCB868

Rooted explant cultures were transformed with the RBM020, RCB873 and RCB868 expression vectors, cultured and inoculated with soybean cyst nematode (SCN) second stage juveniles (J2) as described in Example 4.

Several independent root lines were generated from each binary vector transformation, and the lines were used for bioassay. Four weeks after nematode inoculation, the cysts in each well were counted.

Rooted explant cultures transformed with constructs RMB020, RCB873 and RCB868 exhibited a general trend of reduced cyst numbers and female index compared to the susceptible control. For each of these constructs, the majority of transformed lines had fewer cysts relative to the mean number of cysts found on the susceptible control Williams82.

Overexpression of the soybean pEARLI1-like gene GM50292847 (SEQ ID NO:3) in operative association with the TPP promoter (SEQ ID NO:21), the “superpromoter”, and a second nematode-inducible promoter did not result in a reduction of cyst count. Overexpression of GM4709339 (SEQ ID NO:5), and GM50857725 (SEQ ID NO:7) in operative association with the TPP promoter did not result in a reduction of cyst count.

### Example 7

### Generation of Transgenic Arabidopsis and Fungal and Nematode Bioassays

The Arabidopsis pEARLI1-like gene encoded by At4g12490 is highly homologous to the At4g12500 sequence described above. An RNAi construct comprising nucleotides 1 to 549 of SEQ ID NO:9 (the full-length At4g12490 sequence) was based on the RNAi vector pJawohI8 (GenBank Accession Number: AF408413). This vector contained two GATEWAY-cassettes (Invitrogen, Carlsbad, Calif.) arranged in an inverted orientation separated by an intron (Intron 1 of Arabidopsis thaliana WRKY transcription factor 33, GenBank Accession No NM129404). The RNAi cassette is driven by a CaMV-35S promoter and a 35S polyadenylation signal (terminator) (Zimmerli et al., 2004 Plant Journal 40, 633-46). Transformation of Arabidopsis was performed using standard procedures known to those in the art.

The phytopathogenic fungus Alternaria brassicicola was maintained on 2% Agar containing 2% malt-extract. The plates were incubated at 24° C. with a 12 h light/dark cycle. To inoculate the plants, a spore suspension was prepared by rinsing. 12 day-old fungal culture plates with 2% malt-extract solution and filtering the spore-mycelium suspension was filtered through gauze. The spore density was determined by counting in a Thoma-chamber. A freshly harvested spore suspension with a density of 1-1.5×10⁶ Spores/mL was used to inoculate plants.

The transgenic Arabidopsis plants (ecotype Col-0 and the double mutated Col-0 pen1-pen2 (Lipka et al., Science 310, 1180, 2005; Collins et al., Nature 425, 973, 2003) containing the At4g12490 RNAi construct were grown in soil for approx 26 days under standard 12 hour light conditions.

The plants were inoculated by spraying the spore suspension described above. Immediately after inoculation, the plants were put into the dark with 100% humidity. Forty-eight hours after inoculation the plants were put back in normal conditions, with 12 hours of light and 68% humidity.

The disease symptoms were scored 4 and 8 days after inoculation. Three parameters were used to score the responses of the plants to the pathogen: appearance of mycelium on/in the leaf, appearance of chlorotic areas beneath mycelium, and necrosis and/or maceration of the tissue.

For the dsRNA based on At4g12490 in Arabidopsis Col-0 plants, an enhanced resistance to infection by Alternaria was found compared to wild type controls. All tested plants (13 out of 13 plants) from 5 independent lines showed a reduced level of chlorosis beneath the mycelium. Additionally the area of necrosis was reduced compared to wild type controls. This result demonstrates the involvement of the pEARLI1-like gene At4g12490 in the resistance to the necrotrophic fungus Alternaria brassicicola. For the dsRNA based on At4g12490 in Arabidopsis double mutated Col-0 pen1-pen2 plants, 2 out of 14 plant lines showed a strong chlorosis.

Transgenic plants were also challenged with the biotrophic pathogen H. schactii (beet cyst nematode). In this case, the plants were less resistant than controls. This suggests that modulating the level of pEARLI1-like gene product can influence pathogen resistance.

Those skilled in the art will recognize, or will be able to ascertain using no more than routine experimentation, many equivalents to the specific embodiments of the invention described herein. Such equivalents are intended to be encompassed by the following claims.

1. A transgenic nematode-resistant plant transformed with an expression vector comprising an isolated pEARLI1-like polynucleotide capable of rendering a plant resistant to nematodes.

2. The transgenic plant of claim 1, wherein the isolated pEARLI1-like polynucleotide is selected from the group consisting of:
a) a polynucleotide having a sequence as defined in SEQ ID NO:1, 3, 5, 7, 9, 11, 13, 15, 17, or 19;
b) a polynucleotide encoding a polypeptide having a sequence as defined in SEQ ID NO:2, 4, 6, 8, 10, 12, 14, 16, 18, or 20;
c) a polynucleotide having at least 70% sequence identity to the polynucleotide defined in SEQ ID NO:1, 3, 5, 7, 9, 11, 13, 15, 17, or 19;
d) a polynucleotide encoding a polypeptide having at least 70% sequence identity to the polypeptide defined in SEQ ID NO:2, 4, 6, 8, 10, 12, 14, 16, 18, or 20;
e) a polynucleotide that hybridizes under stringent conditions to the polynucleotide defined in SEQ ID NO:1, 3, 5, 7, 9, 11, 13, 15, 17, or 19; and
f) a polynucleotide that hybridizes under stringent conditions to the polynucleotide encoding the polypeptide defined in SEQ ID NO:2, 4, 6, 8, 10, 12, 14, 16, 18, or 20.

3. The plant of claim 1, further defined as a monocot.

4. The plant of claim 1, further defined as a dicot.

5. The plant of claim 4, wherein the plant is selected from the group consisting of pea, pigeonpea, Lotus, sp., Medicago truncatula, alfalfa, soybean, carrot, celery, tomato, potato, cotton, tobacco, pepper, oilseed rape, beet, cabbage, cauliflower, broccoli, lettuce and Arabidopsis thaliana.

6. A transgenic seed which is true breeding for n expression vector comprising an isolated pEARLI1-like polynucleotide capable of rendering a plant resistant to nematodes.

7. The transgenic seed of claim 6, wherein the isolated pEARLI1-like polynucleotide is selected from the group consisting of:
a) a polynucleotide having a sequence as defined in SEQ ID NO:1, 3, 5, 7, 9, 11, 13, 15, 17, or 19;
b) a polynucleotide encoding a polypeptide having a sequence as defined in SEQ ID NO:2, 4, 6, 8, 10, 12, 14, 16, 18, or 20;
c) a polynucleotide having at least 70% sequence identity to the polynucleotide defined in SEQ ID NO:1, 3, 5, 7, 9, 11, 13, 15, 17, or 19;
d) a polynucleotide encoding a polypeptide having at least 70% sequence identity to the polypeptide defined in SEQ ID NO:2, 4, 6, 8, 10, 12, 14, 16, 18, or 20;
e) a polynucleotide that hybridizes under stringent conditions to the polynucleotide defined in SEQ ID NO:1, 3, 5, 7, 9, 11, 13, 15, 17, or 19; and
f) a polynucleotide that hybridizes under stringent conditions to the polynucleotide encoding the polypeptide defined in SEQ ID NO:2, 4, 6, 8, 10, 12, 14, 16, 18, or 20.

8. A nematode resistance expression vector comprising a promoter operably linked to an isolated pEARLI1-like polynucleotide capable of rendering a plant resistant to nematodes.

9. The expression vector of claim 8, wherein the isolated pEARLI1-like polynucleotide is selected from the group consisting of:
a) a polynucleotide having a sequence as defined in SEQ ID NO:1, 3, 5, 7, 9, 11, 13, 15, 17, or 19;
b) a polynucleotide encoding a polypeptide defined in SEQ ID NO:2, 4, 6, 8, 10, 12, 14, 16, 18, or 20;
c) a polynucleotide having at least 70% sequence identity to the polynucleotide defined in SEQ ID NO:1, 3, 5, 7, 9, 11, 13, 15, 17, or 19;
d) a polynucleotide encoding a polypeptide having at least 70% sequence identity to the polypeptide having the sequence defined in SEQ ID NO:2, 4, 6, 8, 10, 12, 14, 16, 18, or 20;
e) a polynucleotide that hybridizes under stringent conditions to the polynucleotide defined in SEQ ID NO:1, 3, 5, 7, 9, 11, 13, 15, 17, or 19; and
f) a polynucleotide that hybridizes under stringent conditions to a polynucleotide encoding the polypeptide defined in SEQ ID NO:2, 4, 6, 8, 10, 12, 14, 16, 18, or 20.

10. A method of producing a transgenic nematode-resistant plant, the method comprising the steps of:
a) introducing into a plant cell a nematode resistance expression vector comprising a promoter operably linked to an isolated pEARLI1-like polynucleotide capable of rendering a plant resistant to nematodes; and
b) generating from the plant cell the transgenic plant expressing the pEARLI1-like polynucleotide.

11. The method of claim 10, wherein the pEARLI1-like polynucleotide is selected from the group consisting of:
a) a polynucleotide having a sequence as defined in SEQ ID NO:1, 3, 5, 7, 9, 11, 13, 15, 17, or 19;
b) a polynucleotide encoding a polypeptide defined in SEQ ID NO:2, 4, 6, 8, 10, 12, 14, 16, 18, or 20;
c) a polynucleotide having at least 70% sequence identity to the polynucleotide defined in SEQ ID NO:1, 3, 5, 7, 9, 11, 13, 15, 17, or 19;
d) a polynucleotide encoding a polypeptide having at least 70% sequence identity to the polypeptide having the sequence defined in SEQ ID NO:2, 4, 6, 8, 10, 12, 14, 16, 18, or 20;
e) a polynucleotide that hybridizes under stringent conditions to the polynucleotide defined in SEQ ID NO:1, 3, 5, 7, 9, 11, 13, 15, 17, or 19; and
f) a polynucleotide that hybridizes under stringent conditions to a polynucleotide encoding the polypeptide defined in SEQ ID NO:2, 4, 6, 8, 10, 12, 14, 16, 18, or 20.

12. A dsRNA molecule that confers resistance to a necrotrophic fungus to a plant, wherein the dsRNA molecule comprises a first strand substantially identical to a portion of a pEARLI1-like, target gene and a second strand substantially complementary to the first strand.

13. The dsRNA molecule of claim 12, wherein the portion of the pEARLI1-like target gene is from 19 to 500 nucleotides of a sequence selected from the group consisting of:
a) a polynucleotide having a sequence as defined in SEQ ID NO:1, 3, 5, 7, 9, 11, 13, 15, 17, or 19;
b) a polynucleotide encoding a polypeptide having a sequence as defined in SEQ ID NO:2, 4, 6, 8, 10, 12, 14, 16, 18, or 20;
c) a polynucleotide having at least 70% sequence identity to the polynucleotide defined in SEQ ID NO:1, 3, 5, 7, 9, 11, 13, 15, 17, or 19;
d) a polynucleotide encoding a polypeptide having at least 70% sequence identity to the polypeptide defined in SEQ ID NO:2, 4, 6, 8, 10, 12, 14, 16, 18, or 20;
e) a polynucleotide that hybridizes under stringent conditions to the polynucleotide defined in SEQ ID NO:1, 3, 5, 7, 9, 11, 13, 15, 17, or 19; and
h) a polynucleotide that hybridizes under stringent conditions to the polynucleotide encoding the polypeptide defined in SEQ ID NO:2, 4, 6, 8, 10, 12, 14, 16, 18, or 20.

14. A transgenic plant comprising the dsRNA of claim 13, wherein the plant is more resistant to necrotrophic fungus infestation than a wild type plant of the same variety.


SEQUENCE LISTING (21 sequences)

SEQ ID NO: 1 - DNA, 534 bp, Arabidopsis thaliana
atggcttcaaagaactcaacctctcttgctcttttctttgccctcaacatcctttttttcaccttaaccactgctactgattgtcgatgcaacctaagtcctaagcctaggacggtcccaagtccaaaggtcccgagtcctaagtacccaagtccttcgattccaagtccttcggtcccaactccttcagtcccaactccttcagttccaactccttcggtaccaagtcctaaccctacgcctgtcactcctccgagaacccctggttcatccggaaactgtcctatcgatgctctcagactcggtgtgtgtgcgaatgtcctaagtggtctacttaacgtgcagttgggacagccatcagctcaaccatgctgctcgctcatccaaggtttggttgaccttgacgctgcgatttgtctctgcactgcccttagggctaacgttcttggcatcaaccttaacgttcctatatctctcagtgttcttctcaacgtttgcaacagaaggcttccgtctgatttccaatgtgcttaa

SEQ ID NO: 2 - protein, 177 aa, Arabidopsis thaliana
MASKNSTSLALFFALNILFFTLTTATDCRCNLSPKPRTVPSPKVPSPKYPSPSIPSPSVPTPSVPTPSVPTPSVPSPNPTPVTPPRTPGSSGNCPIDALRLGVCANVLSGLLNVQLGQPSAQPCCSLIQGLVDLDAAICLCTALRANVLGINLNVPISLSVLLNVCNRRLPSDFQCA

SEQ ID NO: 3 - DNA, 519 bp, Glycine max
atgggttccaaaactcgctcttcccttgcttttttcctaacagtcaacattctcttctttgcccttgtctctgcctgcgggacatgccctagcccaacgccaaggcataagcacaagcctgttaagccaagcccttcaggtggctcaggtggctcaggaggttcaggtggttcaggaggttctggtggctcaggtggttcaggaggttctggtggctcaggtggttcaggaggttctggtggttctggaggttccggtgcgtcttgccctcgtgatgcactcaaactaggagtgtgtgccaatgtgctaaacgggttgttgaacgtgactttgggtcaaccaccagttaccccatgctgttcccttctcaatggtctcgttgatcttgaagctgcagtgtgcctttgcactgccctaaaagcaaacattttgggcatcaacctcaaccttccaatctcactcagcttacttctcaatgtttgctcaaggaacgccccacgtgatttccaatgtgcctaa

SEQ ID NO: 4 - protein, 172 aa, Glycine max
MGSKTRSSLAFFLTVNILFFALVSACGTCPSPTPRHKHKPVKPSPSGGSGGSGGSGGSGGSGGSGGSGGSGGSGGSGGSGGSGGSGASCPRDALKLGVCANVLNGLLNVTLGQPPVTPCCSLLNGLVDLEAAVCLCTALKANILGINLNLPISLSLLLNVCSRNAPRDFQCA

SEQ ID NO: 5 - DNA, 411 bp, Glycine max
atggcttcaaaaacttgttcctcccttgcccttttcctcacactcaacctcgttttcttctcacttgtctctgcatgcggttatacaccatgccctggtccaaacccaaagccaaggcccaaccccaaccctaaccctaaccccagcccaagtggctcgtgcccccgtgatgcactcaaactaggtgtatgcgccaatgtgctaaacttggtgaacgtcactttgggtcaaccaccagttaccccttgctgcacccttctcgatggtctcgttgaccttgaagctgcagtgtgcctttgcactgccctcaaagcaaacattttgggcatcaacctcaaccttccaatctcactcagcttgcttctcaatgtttgctcaaggaaagtcccacgtgatttccaatgtgcctaa

SEQ ID NO: 6 - protein, 136 aa, Glycine max
MASKTCSSLALFLTLNLVFFSLVSACGYTPCPGPNPKPRPNPNPNPNPSPSGSCPRDALKLGVCANVLNLVNVTLGQPPVTPCCTLLDGLVDLEAAVCLCTALKANILGINLNLPISLSLLLNVCSRKVPRDFQCA

SEQ ID NO: 7 - DNA, 384 bp, Glycine max
atggcttccaagggcacagttgcatccacttatgttgctctattcctttgtctcaacatgctttcctacactatggtgagctccacctacatccctgtaatccctgacccatcagtgccttctcaaaagggtacctgccctatagatgcacttaagttgggtgtgtgtgccaacgtgttgaacttggtcaatgttaaattagggtctccaccaacgctcccatgctgcaacctcattaagggtcttgcggatcttgaagttgctgcatgcctttgcactgccctcaaagctaacgttcttggcatcaacctcaacgtcccaatttccttgagcgtaattctcaacaactgtggaaggaataacgctggcttccagtgcccttga

SEQ ID NO: 8 - protein, 127 aa, Glycine max
MASKGTVASTYVALFLCLNMLSYTMVSSTYIPVIPDPSVPSQKGTCPIDALKLGVCANVLNLVNVKLGSPPTLPCCNLIKGLADLEVAACLCTALKANVLGINLNVPISLSVILNNCGRNNAGFQCP

SEQ ID NO: 9 - DNA, 549 bp, Arabidopsis thaliana
atggcttcaaagaactcagcctctcttgctcttttctttgccctcaacatcctttttttcaccttaaccgctggtactaattgtagatgcaacccgagtcctaagcctaggccgctcccaaatcctaaggtcccaagtcctaaggtcccaactccttcggtcccaagtccatatgttccaactccttcggtcccaagtccttccgtaccaactccttcggtcccaagtccttccgtaccaagtcctaaccctacgccagtcattcctccgagaacccctggttcatccggaaactgtcctatcgatgctctcagactcggtgtatgtgcgaatgtgctaagcggtctacttaacgtgcagttgggacagccatcacctcaaccatgctgctcactcattcaaggtttggttgaccttgacgctgcggtttgcctatgcactgctcttagggctaacgttcttggcatcaaccttaacgttcctatatctctcagtgttcttctcaacgtttgcaacagaaggcttccgtctaatttccaatgtgcttga

SEQ ID NO: 10 - protein, 182 aa, Arabidopsis thaliana
MASKNSASLALFFALNILFFTLTAGTNCRCNPSPKPRPLPNPKVPSPKVPTPSVPSPYVPTPSVPSPSVPTPSVPSPSVPSPNPTPVIPPRTPGSSGNCPIDALRLGVCANVLSGLLNVQLGQPSPQPCCSLIQGLVDLDAAVCLCTALRANVLGINLNVPISLSVLLNVCNRRLPSNFQCA

SEQ ID NO: 11 - DNA, 390 bp, Arabidopsis thaliana
atggcttcaaaaatttcagcctctcttgtcattttcctcacattcaacattctttttttcactttaaccaccgcatgtggcggtggctgcagttccacccctaaacccaaacctaagcccaagtccaccggaagctgccctaaagacactctcaagctcggcgtttgcgccaatgttctcaaggatcttctcaaaattcagttgggaacaccaccagtgaagccttgttgttcgctcctcaatggtttagttgatcttgaggctgccgcttgtctctgcaccgccctaaaggctaaagttttaggcattaatcttaatgttcccgtctctctcagccttcttctcaatgtttgtggcaagaaggttccttccggcttcgtatgtgcttga

SEQ ID NO: 12 - protein, 129 aa, Arabidopsis thaliana
MASKISASLVIFLTFNILFFTLTTACGGGCSSTPKPKPKPKSTGSCPKDTLKLGVCANVLKDLLKIQLGTPPVKPCCSLLNGLVDLEAAACLCTALKAKVLGINLNVPVSLSLLLNVCGKKVPSGFVCA

SEQ ID NO: 13 - DNA, 402 bp, Arabidopsis thaliana
atggctcttaaagactctcttgcgctcctccttctcttcaacatccttttcttcactctcactactgccactcgcagtaccaattgtcctcctccaccaggaaaacacaacaaacaaaaacctagcccaacacccactaccgggacttgtcctaaagatgccttaaaggtcggtgtttgtgttaacgcactcaacttgttaaacggtttgacccccggaactcctcctgtgactccatgttgcagccttatcgagggtttggtagatcttgaagctgcgatttgtctttgcactgctctcaaggctagtgttcttggcatcaaccttacccttcccatcaacctcagcttgcttctcaatatttgcaacagagaggcatcacgtgacttccaattcccttaa

SEQ ID NO: 14 - protein, 133 aa, Arabidopsis thaliana
MALKDSLALLLLFNILFFTLTTATRSTNCPPPPGKHNKQKPSPTPTTGTCPKDALKVGVCVNALNLLNGLTPGTPPVTPCCSLIEGLVDLEAAICLCTALKASVLGINLTLPINLSLLLNICNREASRDFQFP

SEQ ID NO: 15 - DNA, 384 bp, Arabidopsis thaliana
atggcttattctaagattgctcttctcctcatcttcaatgtcatcttcttcactttagtcagctcgacttcagtcccttgtccaccaccaccgcccaagagccaccacaagaaacccgcaacgccatctcctaaacccacttgtaaagacgctcttaaacttaaggtatgtgctaacgtgttggatttggttaaggtttctctgccaccaacgtccaactgttgcgctcttatcaaaggtctagttgatcttgaagccgcggtctgtctttgcactgccctaaaggctaatgttcttggtatcaaccttaatgttcccatttctttgaacgttgtcctaaaccattgtggtaagaaggttccatctggtttcaaatgtgcctag

SEQ ID NO: 16 - protein, 127 aa, Arabidopsis thaliana
MAYSKIALLLIFNVIFFTLVSSTSVPCPPPPPKSHHKKPATPSPKPTCKDALKLKVCANVLDLVKVSLPPTSNCCALIKGLVDLEAAVCLCTALKANVLGINLNVPISLNVVLNHCGKKVPSGFKCA

SEQ ID NO: 17 - DNA, 450 bp, Arabidopsis thaliana
atggcttcaagaaccacaaaatctttagctcttttcttgatcctcaacttccttttcttcacaacaatatccgcctgcggtaactgcggttgcccttctcccaagccaaaacataaacctagcccctctcccaagccaaaacctaaccctaaacccaagccaacaccacaccctagtcctagcccggcaatagccaaatgccctagagacgcccttaagctaggagtttgcgccaacgtactcaacggcctactaaacgtaacccttggcaagccaccggtcgagccatgttgcaccctcatccaaggactcgctgatcttgaggctgcagcttgtctttgcactgcgctcaaggctaacatcctcggtatcaacctgaatatcccactctctctcagtcttcttctcaatgtttgcagcaaaaaagttcctcgtggcttccaatgctaa

SEQ ID NO: 18 - protein, 149 aa, Arabidopsis thaliana
MASRTTKSLALFLILNFLFFTTISACGNCGCPSPKPKHKPSPSPKPKPNPKPKPTPHPSPSPAIAKCPRDALKLGVCANVLNGLLNVTLGKPPVEPCCTLIQGLADLEAAACLCTALKANILGINLNIPLSLSLLLNVCSKKVPRGFQC

SEQ ID NO: 19 - DNA, 354 bp, Arabidopsis thaliana
atgagtatggctccaaagacctcaactacacttgctttattccttgtgaccaatattctcttcctcaacctcattaccctgagttgcgcagacaatacttgcccaagagacgttctcaaactttcgacatgctcgaatgttctcaacctcatcaacttgaagctcggggcaccagctatgaggccttgttgctctattctctttggtctaattgatctcgatgttgctgtttgcctttgcaccgcgctcaagctcagccttcttggcatcaccatcgacactcctattcaccttaacttggctcttaacgcctgtggaggtacccttcctgatggattccgttgcccaacatag

SEQ ID NO: 20 - protein, 117 aa, Arabidopsis thaliana
MSMAPKTSTTLALFLVTNILFLNLITLSCADNTCPRDVLKLSTCSNVLNLINLKLGAPAMRPCCSILFGLIDLDVAVCLCTALKLSLLGITIDTPIHLNLALNACGGTLPDGFRCPT

SEQ ID NO: 21 - DNA, 1999 bp, Arabidopsis thaliana
gtagtgcccttcatggataccaaaagagaaaatttgatttagtgcatacatataacaatataacgccgcataataatactgtataaaacagtcatgtaacgatatgacagcagtaatacagttccaagagacgttataatcgtatgcaatcatatgcttgcgtagattttccaacagttttgtttcgttgataggaggaactcaacactctagggtagtgattggtagacactattagcacaaaaaatattaattttactctgatgtttaccaaaaaagttaccaatcaaatatttaagagatcgtactcttccacggcgactctaaaaaccaaagatataggttagactcataactactttataaagaaaatgtttaacgataactaccgagatctaataaataaaccttcattttcaagtatattatatttgcttcttttgtttatatatcaaaccaagttctggtttataaaaatattagataaaactcgtctaaataggtaggtgtaaaataaaattttaaatttttatcgataatatttaaaatttgaaaagttaataatgatccacacattttttctaatatttaatttagtaatttttgtattaaataaaatttcaatcatatacattcgatttttctatacattttaactatctatttctgcataataaactgtattttcattttatacgcttcatcttatggatgatatttaaattttaaatagtaattcatacactttttaatatttaatttagtattttcttaaatccaaattttaatcttacaatttaaatatctactttaacataatacaaatacaatttaatttcattgtattaaattcaaatataatttgattataataaaatacaatttaattctaaaaagtccatcttagattttaattttcctttttagttttgaaaattaaaaatttaaatttattagatatatatgttactttttcagttttcctatttatttaagaaaaaaatattttttaacacatgtcaacttgtaaacaatagactgaacacgtcattttatattatgtttagttttgaaaattaaagttaattaaatatttatatttcttttttttagcttttctaattatttttaaaatagtaaatatttttaatacaaatcaatatctgaacaatagatttgatacataacataatcctataaattattaacttggaaaacgatagtttatataataaaattattttcttaagttctctaaccataacaattaaactatattttagcgaagaaaagaagagaataccgagagaacgcaacttgcactaaaagctaccactttggcaaatcactcatttatattattatatactatcacctcaattcaatcgaaacctcaaaataacactaatatatacacaaagaaacaacagaataacaccgaagaatataggtttaggaaaatccagaatttgttgagactaaagagatcaaattttcgatacaaggttttgctcaatttgtattttcataataaaattctttatttcaccatagacttacatgattagtttttcttttaataaaaaaaaacacgcgacatgaaaattatattatctcagtgttgtcgaatttgaatttgaattttgagttaaatactacacatttgttgacaacttattaaactttacaagtctgctacaaatattgtcaaatatttactaattaatggaccaaaatcctctaacttgcaaatttgtatctacatcaacttaaaaattaggaatatgcgacccaaaaaaaaaaaaactaggaataataataaaaaaatggaatgatgtggaggaagctctttactctttgagaggaagtttataaattgaccacacatttagtctattatcatcacatgtattaagacttgacaacttgtctttctcacaccaaacccctctcctctgtttcataacatctgctctttcttttttttcctaagccccta